(19) United States
(12) Patent Application Publication
(10) Pub. No.: US 2020/0150125 A1
(43) Pub. Date: May 14, 2020

(54) METHODS OF DIAGNOSING AND PROGNOSING CANCER

(71) Applicant: Yeda Research and Development Co., Ltd., Rehovot (IL)

(72) Inventor: Ayelet EREZ, Moshav Beei Zion (IL)

(73) Assignee: Yeda Research and Development Co., Ltd., Rehovot (IL)

(21) Appl. No.: 16/487,849

(22) PCT Filed: Mar. 12, 2018
(86) PCT No.: PCT/IL2018/050287

§ 371 Date: Aug. 22, 2019

Related U.S. Application Data
(60) Provisional application No. 62/470,276, filed on Mar. 12, 2017.

(51) Int. Cl. G01N 33/574

(52) U.S. Cl. CPC G01N 33/57488; G01N 33/57434

(57) ABSTRACT

Methods of diagnosing cancer are provided. Accordingly there is provided a method of diagnosing cancer in a subject, the method comprising determining a level of urea and/or a pyrimidine synthesis metabolite in a biological sample of the subject, wherein a level of urea below a predetermined threshold; and/or a said level of pyrimidine synthesis metabolite above a predetermined threshold; is indicative of cancer. Also provided are methods of prognosing and treating cancer.

### FIELD AND BACKGROUND OF THE INVENTION

The present invention, in some embodiments thereof, relates to methods of diagnosing and prognosing cancer.

Cancer diagnosis at early stage is essential when it comes to treatment outcome and survival, especially when it conies to highly malignant tumors. Clinically practiced methods for-cancer diagnosis include general well being of the patient, screening tests and medical imaging.

Cancer cells typically undergo metabolic transformations leading to synthesis of biological molecules that are essential for cell division and growth.

The urea cycle (UC) is a metabolic process which converts excess nitrogen derived from the breakdown of nitrogen-containing molecules to the excretable nitrogenous compound - urea. Urea, a colorless, odorless solid which is highly soluble in water and practically non-toxic is the main nitrogen-containing substance in the urine of mammals. Several studies have reported altered expression of specific UC components in several types of cancer and also indicated an association between the pattern of these UC components and poor survival or increased metastasis [see e.g. Chaerkady, R. et al. (2008) J Proteome Res 7, 4289-4298; Lee, Y. Y. et al. (2014) Tumour Biol 35: 1109741105; Syed, N. et al. (2013) Cell Death Dis 4, e458; Miyo et al. (2016) Sci Rep. 6: 38415; Erez et al. (2011) Am J Hum Genet. April 8; 88(4): 402-421; Pavlova et al. (2016) Cell Metab. 23(1): 27-47; Rabinovich, S. et al. (2015) Nature, 527(7578): 379-83; International Patent Application Publication No. WO 2016181393, US Patent Application Publication No. US 20150167094 and U.S. Pat. No. 8,440,184].

International Application Publication No. WO 2016181393 discloses that loss of the UC enzyme argininosuccinate synthetase (ASS1) promotes cancer proliferation by diversion of its substrate aspartate towards CAD enzyme. CAD enzyme, a trifunctional protein comprising carbamoyl-phosphate synthase 2 (CPS2), aspartate transcarbamylase (ATC) and dihydroorotase, mediates the first three reactions in the de-novo synthesis pathway of pyrimidines. Several studies have reported altered expression of CAD in several types of cancer [see e.g. Poliakov et al. (2014) Genet Res Int. 2014: 646193; International Patent Application Publication No. WO 2013096455; and US Patent Application Publication No. US 20140087970].

### SUMMARY OF THE INVENTION

According to an aspect of some embodiments of the present invention there is provided a. method of diagnosing cancer in a subject, the method comprising determining a level of urea and/or a pyrimidine synthesis metabolite in a biological sample of the subject, wherein:

(i) the level of the urea below a predetermined threshold; and/or

(ii) the level of the pyrimidine synthesis metabolite above a predetermined threshold; is indicative of cancer, thereby diagnosing cancer in the subject.

According to some embodiments of the invention, the method comprising determining the level of the urea and the pyrimidine synthesis metabolite and wherein a ratio of the pyrimidine synthesis metabolite level to the urea level above a predetermined threshold is indicative of cancer.

According to an aspect of some embodiments of the present invention there is provided a method of prognosing cancer in a subject, the method comprising determining a level of urea and/or a pyrimidine synthesis metabolite in a biological sample of a subject diagnosed with cancer, wherein:

(i) the level of the urea below a predetermined threshold; and/or

(ii) the level of the pyrimidine synthesis metabolite above a predetermined threshold; is indicative of poor prognosis, thereby prognosing cancer in the subject.

According to some embodiments of the invention, the method comprising determining the level of the urea and the pyrimidine synthesis metabolite and wherein a ratio of the pyrimidine synthesis metabolite level to the urea level above a predetermined threshold is indicative of poor prognosis.

According to an aspect of some embodiments of the present invention there is provided a method of monitoring efficacy of cancer therapy in a subject, the method comprising determining a level of urea and/or a pyrimidine synthesis metabolite in a biological sample of the subject undergoing or following the cancer therapy, wherein:

(i) an increase in the level of the urea; and/or

(ii) a decrease in the level of the pyrimidine synthesis metabolites; from a predetermined threshold or in comparison to the level in the subject prior to the cancer therapy, indicates efficacious cancer therapy.

According to some embodiments of the invention, the method comprising determining the level of the urea and the pyrimidine synthesis metabolite and wherein a decrease in the ratio of the pyrimidine synthesis metabolite level to the urea level from a predetermined threshold or in comparison to the ratio in the subject prior to the cancer therapy, indicates efficacious cancer therapy.

According to some embodiments of the invention, there is provided a method of treating cancer in a subject in need thereof, the method comprising:

(a) diagnosing or prognosing the subject according to the methods of the invention; and wherein when a

(i) level of the urea below a predetermined threshold;

(ii) level of the pyrimidine synthesis metabolite above a predetermined threshold; and/or

(iii) ratio of the pyrimidine synthesis metabolite level to the urea level above a predetermined threshold;
is indicated

(b) treating the subject with a cancer therapy.

According to some embodiments of the invention, there is provided a method of treating cancer in a subject in need thereof, the method comprising:

(a) prognosing the subject according to the method of the invention; and

(b) treating the subject with a cancer therapy according to the prognosis.

According to some embodiments of the invention, there is provided a method of treating cancer in a subject in need thereof, the method comprising:

(a) diagnosing or prognosing the subject according to the method of the invention; and wherein when a

(i) level of the urea below a predetermined threshold;

(ii) level of the pyrimidine synthesis metabolite above a predetermined threshold; and/or

(iii) ratio of the pyrimidine synthesis metabolite level to the urea level above a predetermined threshold;
is indicated

(b) selecting a cancer therapy based on the level of the urea and/or pyrimidine synthesis metabolite.

According to some embodiments of the invention, there is provided a method of treating cancer in a subject in need thereof, the method comprising:

(a) prognosing the subject according to the method of the invention; and

(b) selecting a cancer therapy based on the prognosis.

According to some embodiments of the invention, the biological sample is a biological fluid sample.

According to some embodiments of the invention, the biological fluid sample is selected from the group consisting of urine, blood, plasma, serum, lymph fluid, saliva and rinse fluid that may have been in contact with the tumor.

According to some embodiments of the invention, the biological fluid sample is urine.

According to some embodiments of the invention, the biological fluid sample is selected from the group consisting of blood, plasma and serum.

According to some embodiments of the invention, the biological sample is cell-free.

According to some embodiments of the invention, the biological sample is an in-situ sample.

According to some embodiments of the invention, the predetermined threshold is at least 1.1 fold compared to a control sample.

According to some embodiments of the invention, the control sample is a healthy control sample.

According to some embodiments of the invention, the control sample is a non-cancerous tissue obtained from the subject.

According to some embodiments of the invention, the control sample is a cancerous tissue with urea level and/or pyrimidine synthesis metabolite level similar to the urea level and/or pyrimidine synthesis metabolite level in a healthy tissue of the same type.

According to some embodiments of the invention, the predetermined threshold is at least 1.1 fold.

According to some embodiments of the invention, the method comprising corroborating the diagnosis using a state of the art technique.

According to some embodiments of the invention, the method comprising corroborating the prognosis using a state of the art technique.

According to some embodiments of the invention, the cancer is selected from the group consisting of hepatic cancer, osteosarcoma, breast cancer, colon cancer, thyroid cancer, stomach cancer, lung cancer, kidney cancer, prostate cancer, head and neck cancer, bile duct cancer and bladder cancer.

According to some embodiments of the invention, the cancer is selected from the group consisting of hepatic cancer, osteosarcoma, breast cancer and colon cancer.

According to some embodiments of the invention, the cancer therapy comprises a therapy selected from the group consisting of radiation therapy, chemotherapy and immunotherapy.

According to some embodiments of the invention, the cancer therapy comprises a therapy selected from the group consisting of L-arginine depletion, glutamine depletion, pyrimidine analogs, thymidylate synthase inhibitor and mammalian target of Rapamycin (mTOR) inhibitor.

According to some embodiments of the invention, the cancer therapy comprises an immune modulation agent.

According to some embodiments of the invention, the cancer therapy comprises an agent which induces a pyrimidines to purines nucleotide imbalance.

According to some embodiments of the invention, the immune modulation agent comprises anti-PD1.

According to some embodiments of the invention, the immune modulation agent comprises anti-CTLA4.

According to some embodiments of the invention, the agent which induces a pyrimidines to purines nucleotide imbalance comprises an anti-folate agent.

According to some embodiments of the invention, the anti-folate agent comprises methotrexate.

According to some embodiments of the invention, the pyrimidine synthesis metabolite is selected from the group consisting of Uracil, Thymidine, Orotic acid and Orotidine.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are riot intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-E demonstrate the association between the urea cycle (UC) enzymes and CAD. FIG. 1A is a schematic representation demonstrating that the UC enzymes alternate substrates with CAD. FIG. 1B shows a representative photograph and a bar plot summarizing the crystal violet staining which indicates increased proliferation of cultured fibroblasts extracted from ORNT1 deficient (ORNT1D) or OTC deficient (OTCD) patients as compared to fibroblasts extracted from healthy controls. The Y-axis represents fold change of the staining at 48 hours in comparison to time 0 (P≤0.05, student t-test), n=4 biological repetitions. FIG. 1C is a western blot photograph demonstrating increased levels of CAD and phosphorylated CAD in fibroblasts extracted from ORNT1D and OTCD patients as compared to fibroblasts extracted from healthy patient (NF). FIG. 1D is a plot showing decreased expression of ASS1 and increase expression of SLC25A13 and CAD in fibroblasts extracted from healthy patients following human Cytomegalovirus (CMV) infection as measured by ribosome profiling. Y-axis represents expression normalized to non-infected control. FIG. 1E demonstrates high homology and identity between the UC enzymes and CAD. Protein domain structures were annotated using the NCBI BLAST and conserved domain search server (www(dot)ncbi(dot)nlm(dot)nih(dot)gov/Structure/cdd/wrpsb(dot)cgi). Results show high homology between the proximal UC enzymes proteins CPSI and OTC, and two CAD domains CPS2 and ATC, respectively.

FIGS. 2A-E demonstrate that downregulation of UC enzymes increases cancer proliferation. and pyrimidine synthesis. FIG. 2A is a western blow photograph demonstrating the extent of OTC downregulation using several shRNAs in HepG2 hepatic cancer cell line. FIG. 2B shows a representative photograph and a bar plot summarizing the crystal violet staining which indicates increased proliferation of HepG2 hepatic cancer cells transduced with OTC shRNA, as compared to HepG2 hepatic cancer cell transduced with an empty vector (EV).

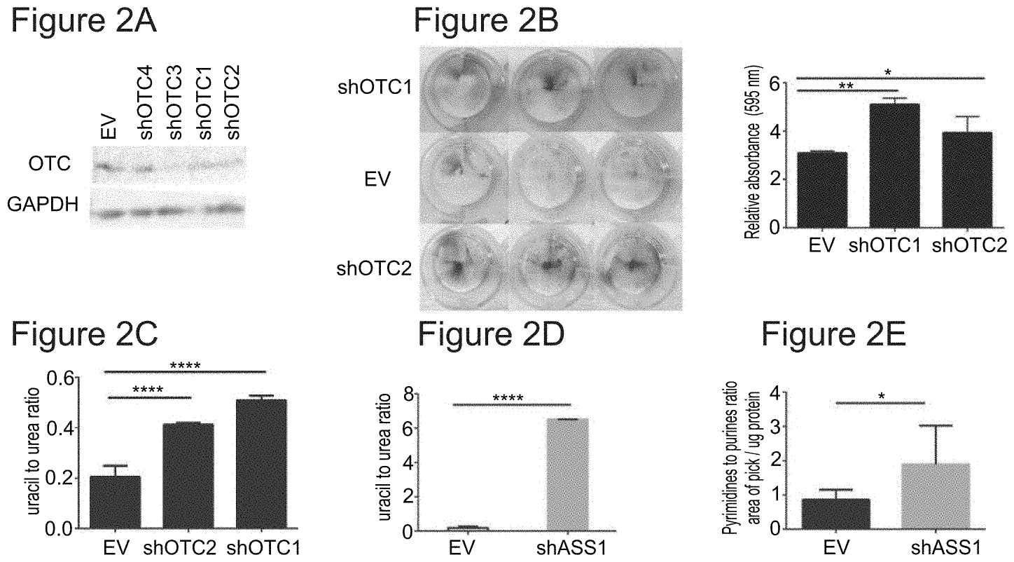

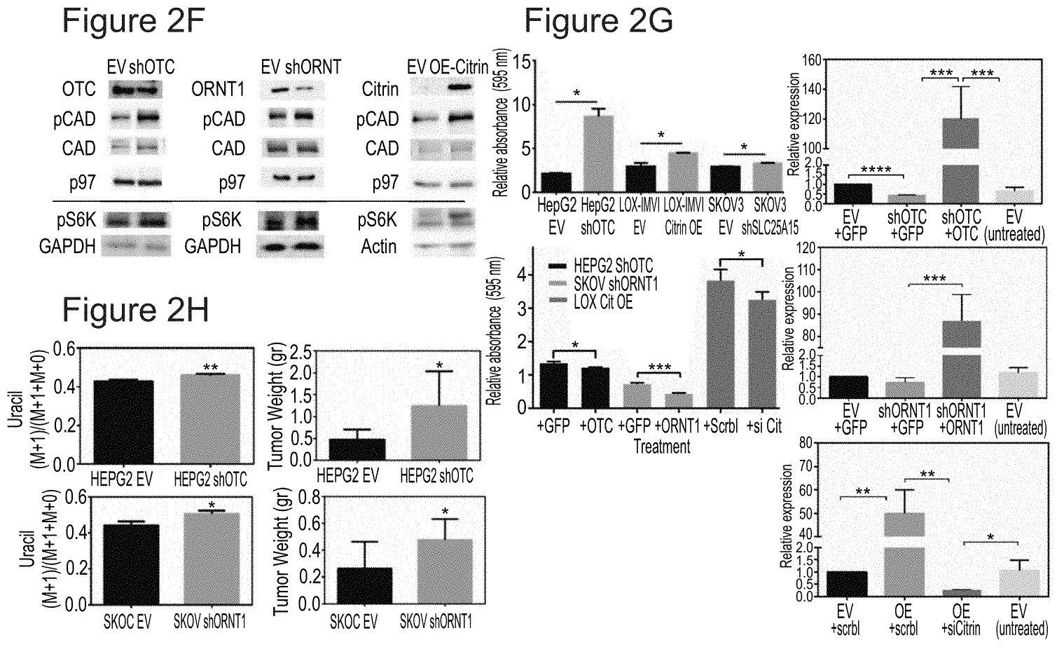

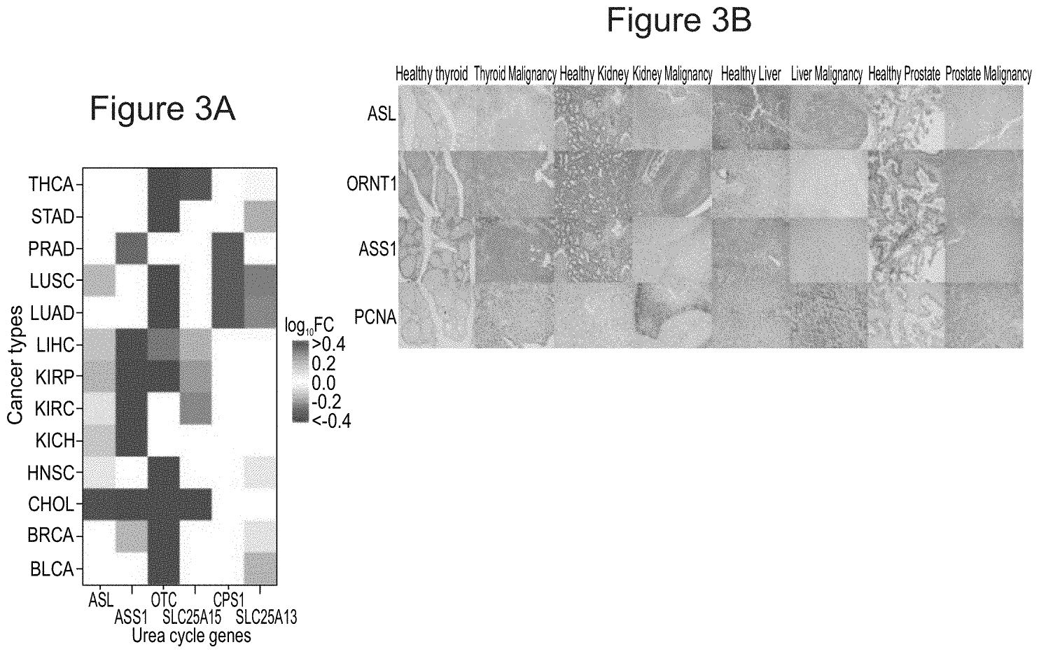

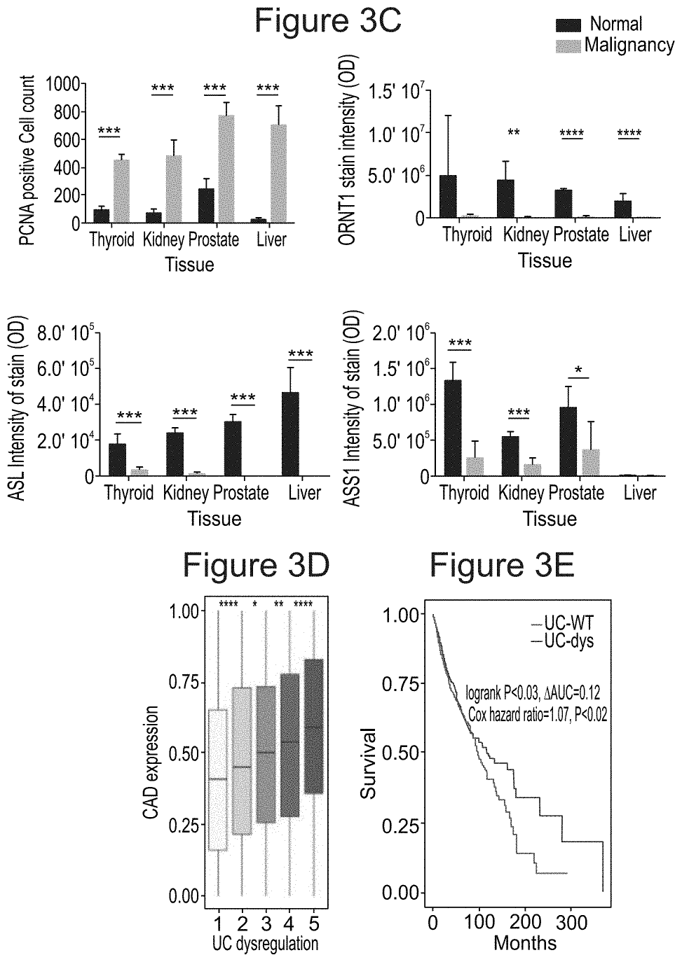

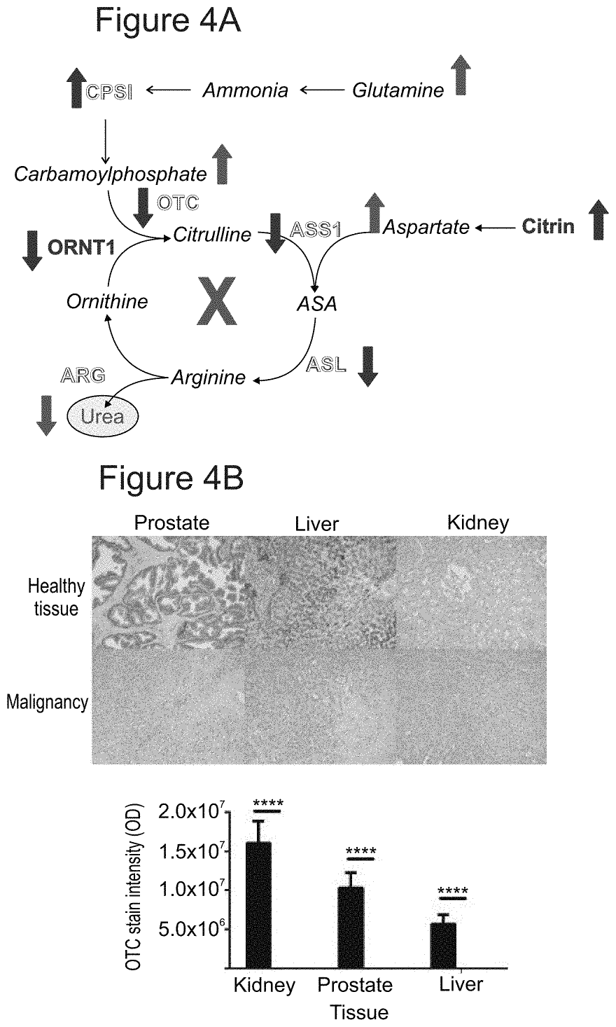

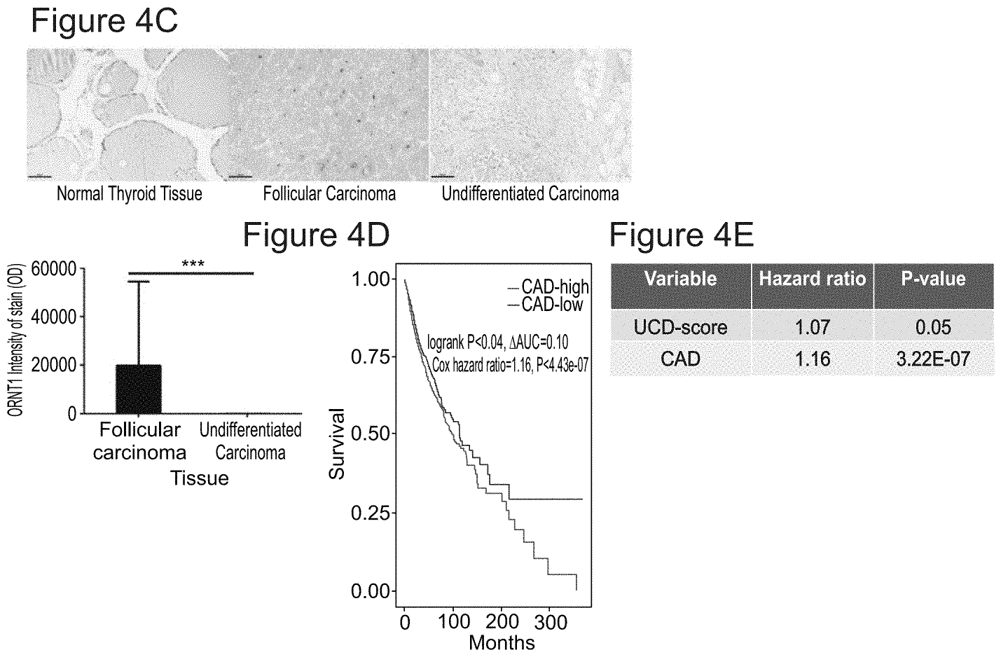

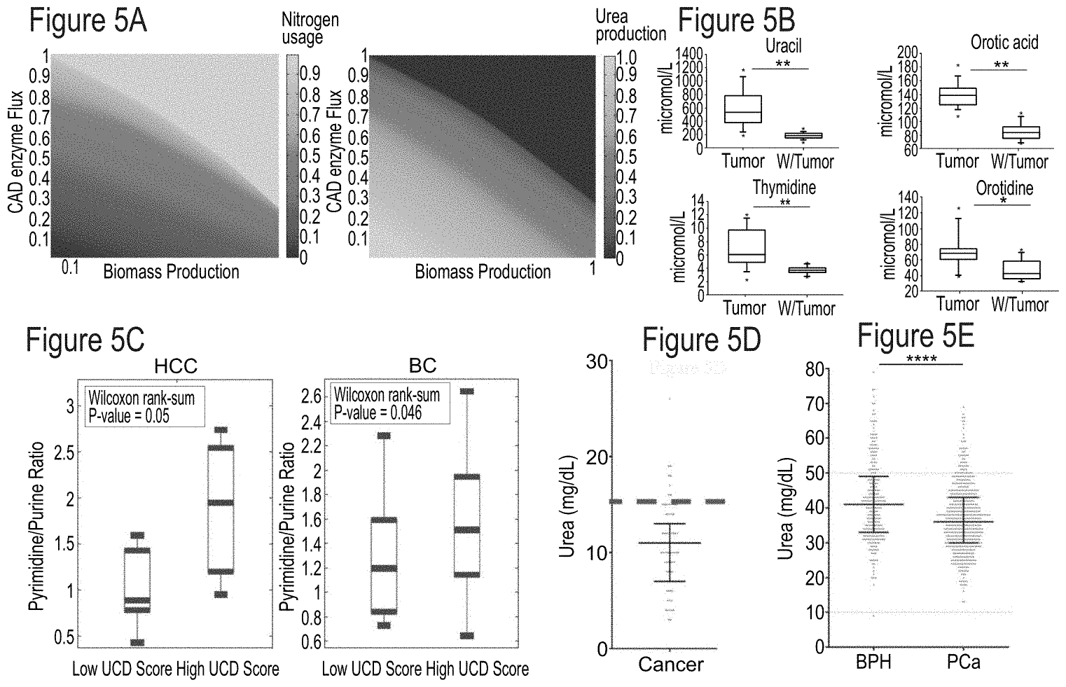

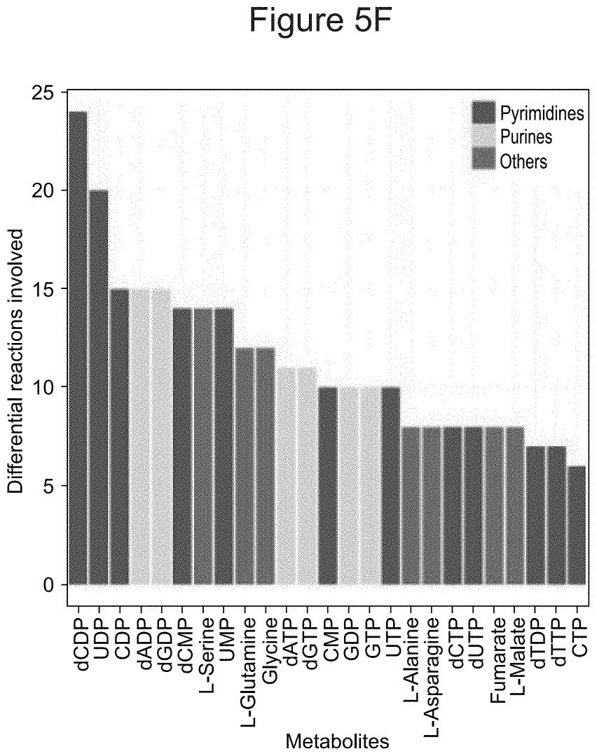

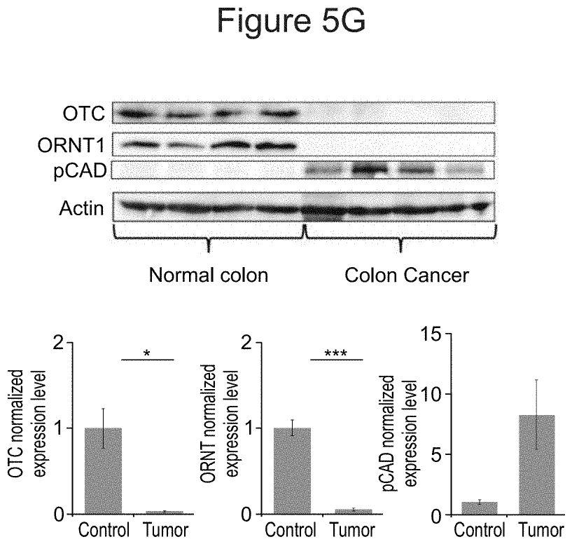

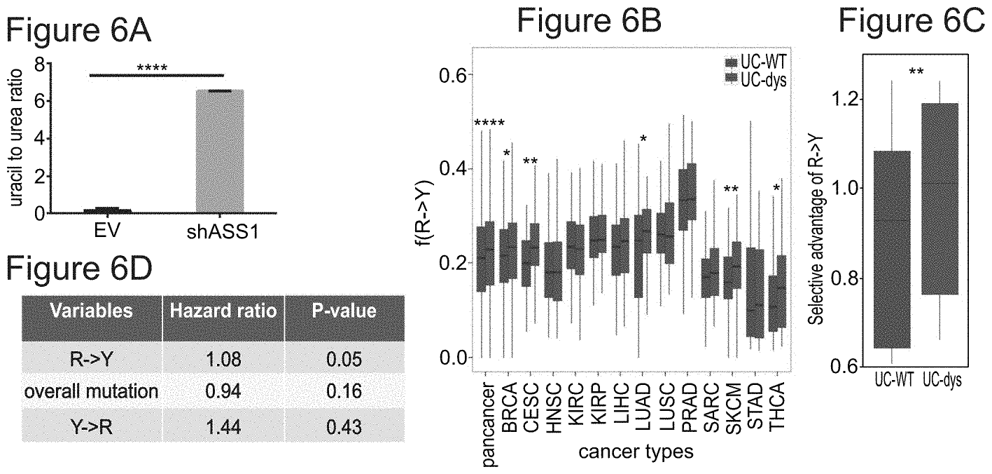

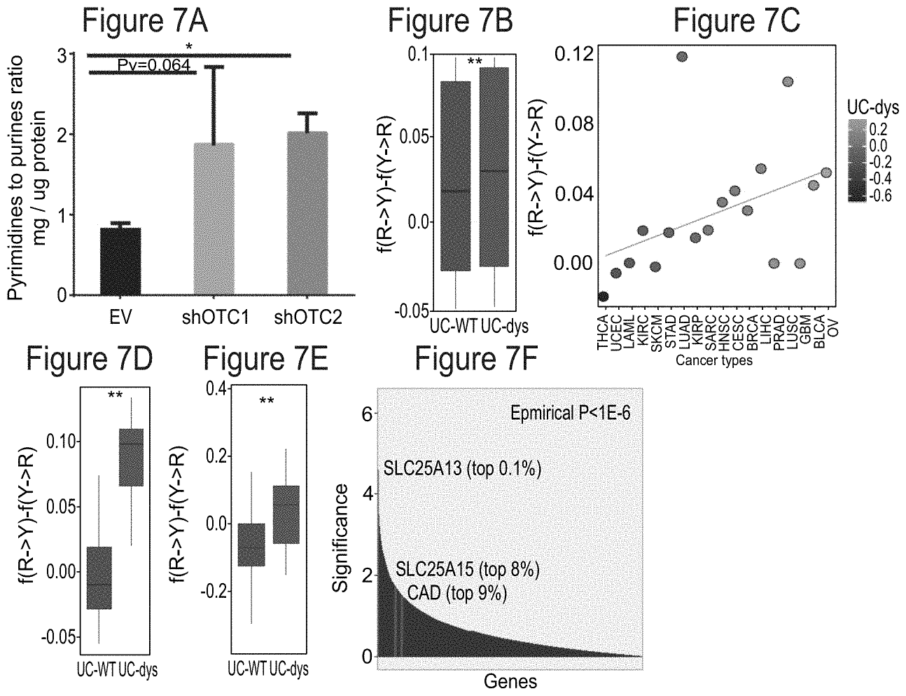

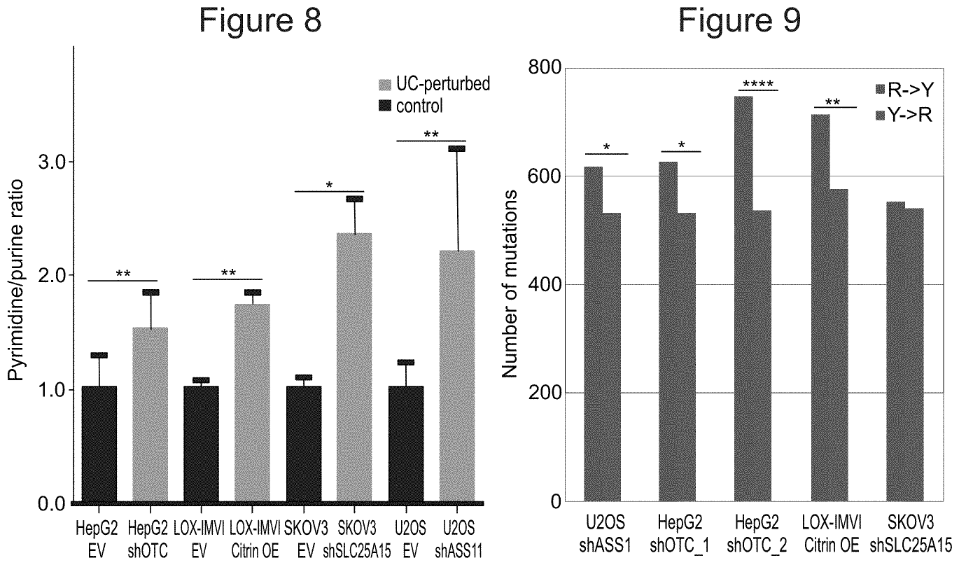

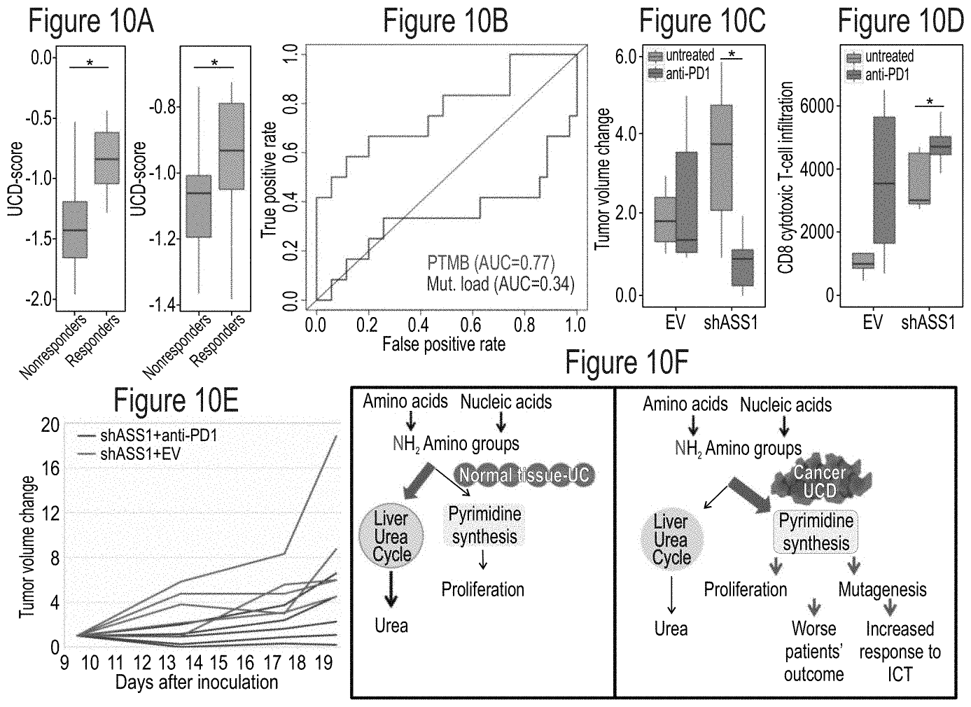

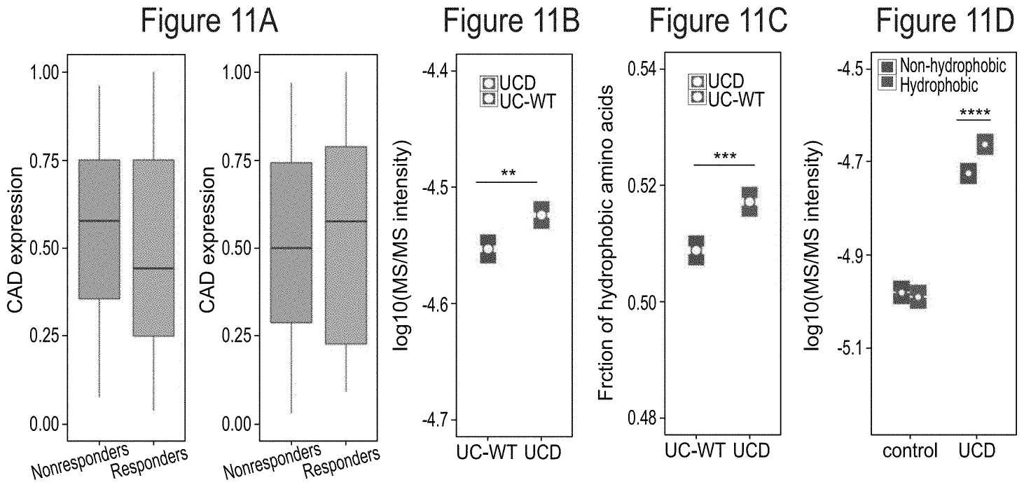

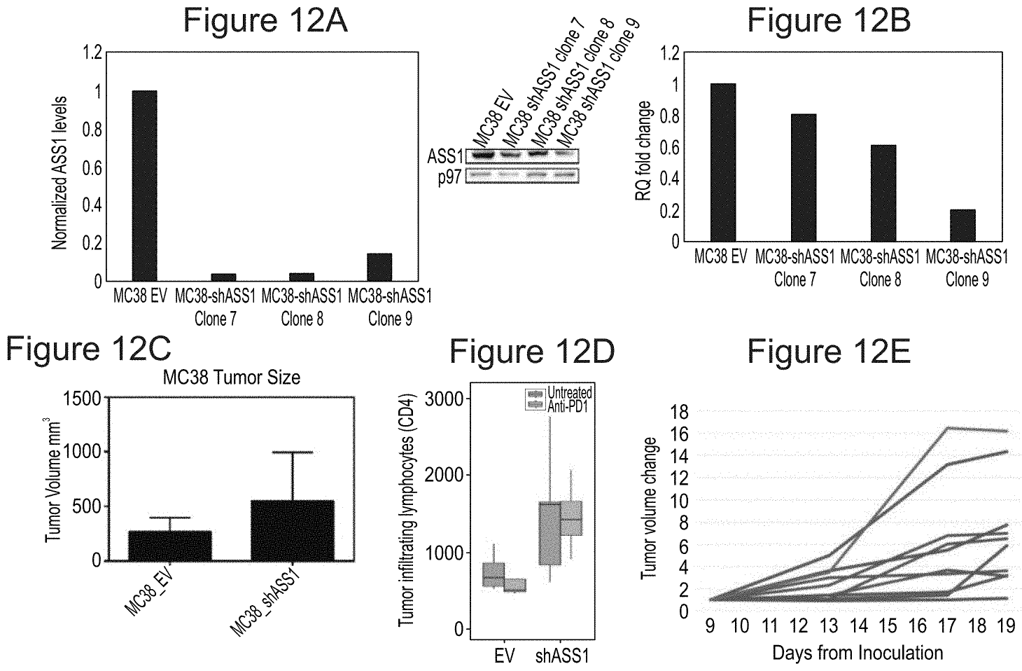

The Y-axis represents fold change of the staining at 48 hours in comparison to time 0 (*P≤0.05, **P≤0.01, student t-test), n=3 biological repetitions. FIG. 2C is a bar plot demonstrating increased uracil to urea ratio in HepG2 hepatic cancer cells transduced with OTC shRNA, as compared to HepG2 hepatic cancer cell transduced with an empty vector (EV), (****P≤0.0001, student t-test), n=3 technical repetitions using GCMS. FIGS. 2D-E are bar plots demonstrating increased uracil to urea ratio (FIG. 2D) and increased pyrimidine to purine ratio in osteosarcoma cells transduced with ASS1 shRNA, as compared to osteosarcoma cells transduced with an empty vector (EV), (*P≤0.05, ****P≤0.0001, student t-test), n=3 biological repetitions.

FIGS. 2F-H demonstrate that specific dysregulation of UC enzymes facilitates cancer proliferation. FIG. 2F shows western blot photographs demonstrating the specific UC perturbations induced in different cancer cells [i.e. downregulation of OTC (shOTC) or ORNT1 (shORNT1) or overexpression of citrin (OE-Citrin)] and the resultant effect on CAD activation compared to control cells transfected with empty vector (EV). FIG. 2G upper left bar plot is a quantification of crystal violet staining showing increased proliferation of different cancer cells following the indicated UC perturbations. FIG. 2G lower left bar plot shows that rescue experiments for the specific UC perturbation reverses the proliferative phenotype. FIG. 2G right bar plots show RT-PCR quantification for the changes in UC genes RNA expression levels following transfection with the specific rescue plasmid versus control plasmids. FIG. 2H left bar plots show enhanced synthesis of labelled M+1 uracil from 15N-a-glutamine in HepG2 cancer cells transduced with OTC shRNA and SKOV cancer cells transduced with ORNT1 shRNA as compared to controls transduced with empty vector. FIG. 2H right bar plots show in vivo growth of HepG2 transduced with OTC shRNA and SKOV transduced with ORNT1 shRNA xenografts compared to xenografts transduced with an empty vector.

FIGS. 3A-E demonstrate that dysregulation of the UC genes (denoted herein as UCD) in cancer activates CAD and correlates with worse prognosis. FIG. 3A shows relative expression of 6 UC genes in tumors from the cancer genome atlas (TCCA) with respect to their expression in healthy control tissues. Most tumors have aberrant expression of at least 2 UC components in the direction that metabolically supplies the required substrates for CAD activity [that is, decreased expression of ASL, ASS1, OTC and/or ONRT1D (SLC23A15) and/or increased expression of CPS1 and/or SLC25A13, P<2.67E-3]. Tumor type's abbreviations are as follows: THCA—Thyroid cancer, STAD—Stomach adenocarcinoma, PRAD—Prostate cancer, LUSC—Lung squamous carcinoma, HNC—Liver hepatocellular carcinoma, KIRP—Kidney renal papillary cell carcinoma, KIRC—Kidney renal Clear Cell Ca, KWH—Kidney chromophobe, HNSC—Head Neck Squamous Cell Carcinoma, CHOL cholangiocarcinoma, BRCA—breast cancer, BLCA—Bladder cancer. FIG. 3B shows immunohistochemistry images of cancer tissues with their respective healthy tissue controls stained with the indicated UC components or PCNA as a marker for proliferation, showing inverse correlation between the expression of UC genes and the proliferation marker. Magnification ×10. FIG. 3C shows bar plots summarizing staining intensity of the PCNA positive cell count and UC proteins. Each staining was calibrated and repeated in two technical repetitions per patient sample in each slide (intensity OD level was compared in a matched T-student test). FIG. 3D is a graph demonstrating that UCD-scores (X-axis, equally divided into 5 bins) are positively correlated with CAD expression. Each paired consecutive bins were compared using the Wilcoxon rank sum test. FIG. 3E is a Kaplan-Meier survival curve showing that UCD is associated with worse survival of patients computed across all TCGA samples (i.e. pan cancer analysis)

FIGS. 4A-E demonstrate that UCD in cancer correlates with tumor grade. FIG. 4A is a schematic representation demonstrating the direction of UC enzymes expression that supports CAD activation (represented in blue arrows). The resulting changes in metabolites' levels following these expression alterations are represented by red arrows. FIG. 4B shows immunohistochemistry images of cancer tissues with their respective healthy tissue controls stained with OTC Magnification ×10; and a bar plot summarizing OTC staining intensity. Each staining was calibrated and repeated in 2 technical repetitions per patient sample in each slide (intensity OD level was compared in a matched T-student test, ****P≤0.0001), FIG. 4C shows immunohistochemistry images of thyroid cancer tissues stained with ORNT1 Magnification ×10; and a bar plot summarizing ORNT1 staining intensity; demonstrating that low levels of ORNT1 are associated with more advanced thyroid tumor grades. Each staining was calibrated and repeated in 2 technical repetitions per patient sample in each slide (intensity OD level was compared in a matched T-student test, ***P≤0.001). FIG. 4D is a Kaplan-Meier survival curve showing that CAD is associated with worse survival of patients computed across all TCGA samples (i.e. pan cancer analysis). FIG. 4E shows a Cox regression analysis of the UCD-score and CAD expression, demonstrating that both variables are independently significant.

FIGS. 5A-G demonstrate that UCD in cancer increases nitrogen utilization. FIG. 5A shows metabolic modelling which predicts decreased urea excretion (left panel) and increased nitrogen utilization (right panel) with increased CAD activity, at high biomass production (that is, higher cell proliferation) conditions. FIG. 5B shows bar plots demonstrating increased pyrimidine pathway metabolites' in urine of breast or colon tumors bearing mice (n=37) as compared to control mice (W/Tumor); n=11), (*P<0.05, **P<0.01, Mann-Whitney test). FIG. 5C shows plots demonstrating the distribution of the ratio of pyrimidine to purine metabolites for samples with low and high UCD-scores (top and bottom 15%). The plot on the left shows the results for hepatocellular carcinoma (HCC) tumors and the plot on the right for Breast cancer (BC) tumors. FIG. 5D is a plot showing urea plasma levels in children with different cancers.

The dashed red line demonstrates the normal age matched mean urea value. FIG. 5E is a plot showing urea plasma levels in patients with prostate cancer (PCa, n=519) as compared to age matched patients with benign prostate hyperplasia (BPH, n=257), ****P<0.0001, Mann-Whitney test. FIG. 5F shows metabolic modelling which predicts a significant increase in metabolic flux reactions involving pyrimidine metabolites following UCD. FIG. 5G shows western blot photographs and their quantification bar plots demonstrating that the increased pyrimidine pathway metabolites' in urine of colon tumors bearing mice shown in FIG. 5B correlates with UCD in the tumors compared to control healthy colon.

FIGS. 6A-D demonstrate that tumors with UCD have increased transverse coding mutations. FIG. 6A is a bar plot demonstrating that downregulation of ASS1 in osteosarcoma cancer cells using shRNA increases pyrimidine to purines ratio as compared to osteosarcorna cancer cells transduced with an empty vector (EV), (****P-value<0.0001, two way ANOVA with Dunnett's correction). FIG. 6B is a plot demonstrating that UCD (UC-dys) increases DNA purine to pyrimidine transversion mutations at a pan-cancer scale and across different tumor types compared to tumors with intact UC (UC-WT). FIG. 6C is a plot demonstrating that UCD samples show a higher fraction of nonsynonymous purine to pyrimidine transversion mutations as compared to UC-WT across all TCGA data (P<4.93E-3). Such a significant bias is riot present for any of the other transversion mutation types (Y->Y, R->R, and Y->R). FIG. 6D shows a Cox regression analysis demonstrating that only R->Y mutation levels are significantly associated with survival (while overall mutation levels and Y->R mutation levels are not).

FIGS. 7A-F demonstrate that UCD increases transversion mutations in tumors. FIG. 7A is a bar plot demonstrating that downregulation of OTC in hepatic cancer cells using shRNA increases pyrimidine to purines ratio as compared to hepatic cancer cells transduced with an empty vector (EV), as measured by LCMS Bars represent the mean of >2 biological repeats, *P<0.05, one way anova with dunnet correction. FIG. 7B is a plot demonstrating that tumors with UCD (UC-dys) have significantly higher number of transversion mutations from purines to pyrimidines on the coding (sense) DNA strand versus tumors with intact UC (UC-WT), Wilcoxon rank sum P<2.35E-3), while such a significance is not observed for transition mutations. FIG. 7C is a plot demonstrating that UCD is associated with higher number of purine to pyrimidine transversion mutations across different cancer types [each circle denotes the UCD and transversion mutation bias levels in a given cancer type, (overall Spearman correlation=0.58, P<0.01]. FIG. 7D is a plot demonstrating that tumors with UCD have significantly greater fractions of transversion mutations from purines to pyrimidines at the mRNA level, based on 18 breast cancer samples (Wilcoxon rank sum, **P<0.001). Only those variants that were detected as a somatic mutation in the exome sequence and were mapped in the corresponding RNA sequence were considered. FIG. 7E is a plot representing genome wide proteomic analysis of 42 breast cancers demonstrating a significantly increased R->Y mutation rates in UCD tumors as compared to tumors with intact UC (Wilcoxon rank sum P<0.02). FIG. 7F is a plot demonstrating that CAD, SLC25A13 and SLC25A15 genes' expression are among the top 10% of genes that correlate most strongly with DNA purines to pyrimidines transversion mutations.

FIG. 8 is a bar plot demonstrating that specific UC perturbations induced in different cancer cells [i.e. downregulation of OTC (shOTC), ORNT1 (shSLC25A15) or ASS1 (shASS1) or overexpression of citrin (Citrin OE)] increases pyrimidine to purines ratio as compared to control cancer cells transduced with an empty vector (EV), as measured by LCMS. Shown is a representative of the mean of more than two biological repeats. (*P≤0.05, **P≤0.01, one way ANOVA with Dunnet's correction).

FIG. 9 is a bar graph demonstrating that specific UC perturbations induced in different cancer cells [i.e. downregulation of OTC (shOTC), ORNT1 (shSLC25A15) or ASS1 (shASS1) or overexpression of citrin (Citrin OE)] increases purines to pyrimidines (R->Y) mutations using a Fisher's exact test.

FIGS. 10A-F demonstrate that UCD score correlates with response to immune modulation therapy (ICT). FIG. 10A demonstrates that UCD-scores are significantly higher in human patients responding to anti-PD1 (left panel) and anti-CTLA4 (right panel) therapies (orange) compared to non-responders (grey) (Wilcoxon ranksum P<0.05). FIG. 10B shows ROC curves demonstrating higher predictive power of pyrimidine-rich transversion mutational bias (PTMB, AUC=0.77, blue) compared to mutational load (AUC-0.34, red) in predicting the response to anti-PD1 therapy (Roh et al., 2017). FIGS. 10C-E demonstrates that anti-PD1 therapy is more efficient in UCD tumors, as determined in an in-vivo syngeneic mouse model of colon cancer. Specifically, control MC-38 mouse colon cancer cells (EV) or MC-38 mouse colon cancer cells transduced with ASS1 shRNA (shASS1) were inoculated into C57BL6 mice injected intraperitoneally with anti-PD1 immunotherapy (N=20 mice, 5 mice in each group). FIG. 10C demonstrates tumor volume 22 days following inoculation (Wilcoxon ranksum P<0.007). FIG. 10D shows CD8 T cells infiltration in the tumors excised on day 21 following inoculation, as evaluated by flow cytometry analysis (Wilcoxon ranksum P=0.01 and 0.3, respectively for shASS1 and EV). FIG. 10E demonstrates tumor growth over time in the shASS1 group with or without anti-PD1 (P<0.01, ANOVA with Dunnett's correction). FIG. 10F is a schematic representation summary the “UCD effect”: while in normal tissues excess nitrogen is disposed as urea, in cancer cells most nitrogen is utilized for synthesis of macromolecules, with pyrimidine synthesis playing a major role in carcinogenesis and effecting patients' prognosis and response to ICT.

FIGS. 11A-D demonstrate the impact of CAD and PTMB on ICT response and HLA-peptide presentation. FIG. 11A demonstrates the expression of CAD is less associated with ICT response than UCI) both in anti-PD1 (Hugo et al., 2016) (left panel) and anti-CTLA4 (Van Allen et al., 2015) (right panel) cohort (Wilcoxon ranksum P=0.71 and 0.45, respectively). FIG. 11B shows peptidomics analysis which demonstrates that UCD cell lines have higher MS/MS intensity than control cell lines (Wilcoxon rariksum P<0.001). FIG. 11C demonstrates that UCD cell lines have more hydrophobic peptides than control cell lines (Wilcoxon ranksum P<0.0002). FIG. 11D demonstrates that hydrophobic peptides (hydrophobicity score >80-percentile) are more abundant (MS/MS intensity) than non-hydrophobic peptides (hydrophobicity-score <20-pervcentile) in UCI) cell lines Vilcoxon ranksum P<1 E-6) but not in control cell lines (Wilcoxon ranksum P=0.14).

FIGS. 12A-E demonstrates that UCD perturbed mouse colon cancers respond better to ICT. FIG. 12A shows western blot photograph and a quantification bar graph demonstrating that MC-38 mouse colon cancer cells infected with different shASS1 clones demonstrate downreguiation of ASS1 at the protein level as compared to control cells infected with an empty vector (EV). FIG. 12B is a RT PCR quantification bar graph demonstrating decreased ASS1 levels in MC38 infected with different shASS1 clones as compared to MC38 infected with EV. FIG. 12C is a bar graph demonstrating that in vivo tumor growth was enhanced in MC38 transduced with shASS1 as compared to the growth of MC38-EV tumors 22 days following inoculation. FIG. 12D shows CD4 T cells infiltration in the tumors excised on day 22 following inoculation, as evaluated by flow cytometry analysis (N=20 mice. 5 mice in each group, Wilcoxon ranksum P>0.4 both for shASS1 and EV). FIG. 12E demonstrates tumor growth over time in the control group (EV) with (red) or without (blue) anti-PD1 (ANOVA P>0.12).

### DESCRIPTION OF SPECIFIC EMBODIMENTS OF THE INVENTION

The present invention, in some embodiments thereof, relates to methods of diagnosing and prognosing cancer.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Cancer cells typically undergo metabolic transformations leading to synthesis of biological molecules that are essential for cell division an d growth.

Whilst reducing the present invention to practice, the present inventors have now uncovered that changes in nitrogen composition (urea and pyrimidine synthesis metabolites) in cancer patients' biofluids are indicative of cancer diagnosis and prognosis.

As is illustrated hereinunder and in the examples section, which follows, the present inventors present several computational modeling and experimental studies of urine and plasma samples, which show increased levels of pyrimidine synthesis metabolites (Uracil, Thymidine, Orotic acid and Orotidine) and decreased levels of urea in urine and plasma samples of tumor bearing mice and cancer patients, respectively, compared to cancer-free mice and patients (Example 3, FIGS. 5A-B, 5D-E).

Consequently, according to some embodiments, decreased levels of urea and increased levels of pyrimidine synthesis metabolites in biological samples, such as urine and plasma, can be used as markers for diagnosing, prognosing and treating cancer.

Thus, according to a first aspect of the present invention, there is provided a method of diagnosing cancer in a subject, the method comprising determining a level of urea and/or a pyrimidine synthesis metabolite in a biological sample of the subject, wherein:

(i) said level of said urea below a predetermined threshold; and/or

(ii) said level of said pyrimidine synthesis metabolite above a predetermined threshold;

is indicative of cancer, thereby diagnosing cancer in the subject.

As used herein the phrase “diagnosing” refers to classifying a pathology (e.g., cancer) or a symptom, determining a severity of the pathology, monitoring pathology progression, forecasting an outcome of a pathology and/or prospects of recovery.

As the teachings of the present invention indicate that low levels of urea and high levels of pyrimidine synthesis metabolites in biological samples of subjects indicate higher tumor grade and decreased survival, the methods of the present invention can be used for prognosing cancer.

Thus, according to an aspect of the present invention, there is provided a method of prognosing cancer in a subject, the method comprising determining a level of urea and/or a pyrimidine synthesis metabolite in a biological sample of a subject diagnosed with cancer, wherein:

(i) said level of said urea below a predetermined threshold; and/or

(ii) said level of said pyrimidine synthesis metabolite above a predetermined threshold;

is indicative of poor prognosis, thereby prognosing cancer in the subject.

Thus, a decreased level of urea, an increased level of a pyrimidine synthesis metabolite is indicative of poor prognosis and/or an increased ratio of a pyrimidine synthesis metabolite level to urea level is indicative of cancer and/or poor prognosis. On the other hand, no change in the metabolites levels, or an increased level of urea, a decreased level of the pyrimidine synthesis metabolite and/or a decreased ratio of a pyrimidine synthesis metabolite level to urea level, indicates better prognosis.

As used herein the term “prognosing” refers to determining the outcome of the disease (cancer).

As used herein “poor prognosis” refers to increased risk of death due to the disease, increased risk of progression of the disease (e.g. cancer grade), and/or increased risk of recurrence of the disease.

As used herein the term “subject” refers to a mammal(e.g., human being) at any age or of any gender.

According to specific embodiments, the subject is a human subject.

According to specific embodiments, the subject is diagnosed with a disease cancer) or is at risk of developing a disease (i.e. cancer).

According to specific embodiments, the subject is not afflicted with an ongoing inflammatory disease (other than cancer).

According to specific embodiments, the subject is not a pregnant female.

Cancers which may be diagnosed, prognosed, monitored or treated by some embodiments of the invention can be any solid or non-solid cancer and/or cancer metastasis. Examples of cancer include but are not limited to, carcinoma, lymphoma, blastoma, sarcoma, and leukemia.

More particular examples of such cancers include, but not limited to, tumors of the gastrointestinal tract (colon carcinoma, rectal carcinoma, colorectal carcinoma, colorectal cancer, colorectal adenoma, hereditary nonpolyposis type 1, hereditary nonpolyposis type 2, hereditary nonpolyposis type 3, hereditary nonpolyposis type 6; colorectal cancer, hereditary nonpolyposis type 7, small and/or large bowel carcinoma, esophageal carcinoma, tylosis with esophageal cancer, stomach carcinoma, pancreatic carcinoma, pancreatic endocrine tumors), endometrial carcinoma, dermatofibrosarcoma protuberans, gallbladder carcinoma, Biliary tract tumors, prostate cancer, prostate adenocarcinoma, renal cancer (e.g., Wilms' tumor type 2 or type 1), liver cancer (e.g., hepatoblastoma, hepatocellular carcinoma, hepatocellular cancer), bladder cancer, embryonal rhabdomyosarcoma, germ cell tumor, trophoblastic tumor, testicular germ cells tumor, immature teratorna of ovary, uterine, epithelial ovarian, sacrococcygeal tumor, choriocarcinoma, placental site trophoblastic tumor, epithelial adult tumor, ovarian carcinoma, serous ovarian cancer, ovarian sex cord tumors, cervical carcinoma, uterine cervix carcinoma, small-cell and non-small cell lung carcinoma, nasopharyngeal, breast carcinoma (e.g., ductal breast cancer, invasive intraductal breast cancer, sporadic; breast cancer, susceptibility to breast cancer, type 4 breast cancer, breast cancer-1, breast cancer-3; breast-ovarian cancer), squamous cell carcinoma (e.g., in head and neck), neurogenic tumor, astrocytoma, ganglioblastoma, neuroblastoma, lymphomas (e.g., Hodgkin's disease, non-Hodgkin's lymphoma, B cell, Burkitt, cutaneous T cell, histiocytic, lymphoblastic, T cell, thymic), gliomas, adenocarcinoma, adrenal tumor, hereditary adrenocortical carcinoma, brain malignancy (tumor), various other carcinomas (e.g., bronchogenic large cell, ductal, Ehrlich-Lettre ascites, epidermoid, large cell, Lewis lung, medullary, mucoepidermoid, oat cell, small cell, spindle cell, spinocellular, transitional cell, undifferentiated, carcinosarcoma, choriocarci noma, cystadenocarcinoma), ependimoblastoma, epithelioma, erythroleukemia (e.g., Friend, lymphoblast), fibrosarcoma, giant cell tumor, glial tumor, glioblastoma (e.g., multiforme, astrocytoma), glioma hepatoma, heterohybridoma, heteromyeloma, histiocytoma, hybridoma (e.g., B cell), hypernephroma, insulinoma, islet tumor, keratoma, leiomyoblastoma, leiomyosarcoma, leukemia (e.g., acute lymphatic, acute lymphoblastic, acute lymphoblastic pre-B cell, acute lymphoblastic T cell leukemia, acute-megakaryoblastic, monocytic, acute myelogenous, acute myeloid, acute myeloid with eosinophilia, B cell, basophilic, chronic myeloid, chronic, B cell, eosinophilic, Friend, granulocytic or myelocytic, hairy cell, lymphocytic, megakaryoblastic, monocytic, monocytic-macrophage, myeloblastic, myeloid, myelomonocytic, plasma cell, pre-B cell, promyelocytic, subacute, T cell, lymphoid neoplasm, predisposition to myeloid malignancy, acute nonlymphocytic leukemia), lymphosarcoma, melanoma, mammary tumor, mastocytoma,, medulloblastoma, mesothelioma, metastatic tumor, monocyte tumor, multiple myeloma, myelodysplastic syndrome, myeloma, nephroblastoma, nervous tissue glial tumor, nervous tissue neuronal tumor, neurinoma, neuroblastoma, oligodendroglioma, osteochondroma, osteomyeloma, osteosarcoma (e.g., Ewing's), papilloma, transitional cell, pheochromocytoma, pituitary tumor (invasive), plasmacytoma, retinoblastoma, rhabdomyosarcoma, sarcoma (e.g., Ewing's, histiocytic cell, Jensen, osteogenic, reticulum cell), schwannoma, subcutaneous tumor, teratocarcinoma (e.g., pluripotent), teratoma, testicular tumor, thymoma and trichoepithelioma, gastric cancer, fibrosarcoma, glioblastoma multiforme; multiple glomus tumors, Li-Fraumeni syndrome, liposarcoma, lynch cancer family syndrome II, male germ cell tumor, mast cell leukemia, medullary thyroid, multiple meningioma, endocrine neoplasia myxosarcoma, paraganglioma, familial nonchromaffin, pilomatricoma, papillary, familial and sporadic, rhabdoid predisposition syndrome, familial, rhabdoid tumors, soft tissue sarcoma, and Turcot syndrome with glioblastoma.

According to specific embodiments, the cancer is carcinoma.

According to specific embodiments, the cancer is not thyroid cancer.

According to specific embodiments, the cancer is not hepatocellular carcinoma.

According to specific embodiments, the cancer is selected from the list of cancers presented in FIG. 3A, each possibility represents a separate embodiment of the present invention.

According to specific embodiments, the lung cancer is lung squamous carcinoma.

According to specific embodiments, the liver cancer is liver hepatocellular carcinoma.

According to specific embodiments, the kidney cancer is kidney renal papillary cell carcinoma.

According to specific embodiments, the kidney cancer is kidney renal clear cell carcinoma.

According to specific embodiments, the kidney cancer is Kidney chromophobe.

According to specific embodiments, the head and neck cancer is Head Neck Squamous Cell Ca.

According to specific embodiments, the bile duct cancer is cholangiocarcinoma.

According to specific embodiments, the cancer is selected from the group consisting of hepatic cancer, osteosarcoma, breast cancer, colon cancer, thyroid cancer, stomach cancer, lung cancer, kidney cancer, prostate cancer, head and neck cancer, bile duct cancer and bladder cancer, each possibility represents a separate embodiment of the present invention.

According to specific embodiments, the cancer is selected from the group consisting of hepatic cancer, osteosarcoma, breast cancer and colon cancer, each possibility represents a separate embodiment of the present invention.

As noted, the methods of the present invention comprise determining a level of urea and/or a pyrimidine synthesis metabolite in a biological sample of the subject.

The phrase “biological sample” as used herein refers to any cellular or non-cellular biological samples which may contain urea and/or a pyrimidine synthesis metabolite. Examples include but are not limited to, a blood sample, a serum sample, a plasma sample, a urine sample, lymph fluid, saliva, rinse fluid that may have been in contact with the tumor, a tissue biopsy, a tissue and an organ.

According to specific embodiments, the biological sample used by the methods of the present invention is a biological fluid sample.

According to specific embodiments, the biological fluid sample is selected from the group consisting of urine, blood, plasma, serum, lymph fluid, saliva and rinse fluid that may have been in contact with the tumor, each possibility represents a separate embodiment of the present invention.

According to specific embodiments, the biological fluid sample is urine.

According to specific embodiments, the biological fluid sample is selected from the group consisting of blood, plasma and serum, each possibility represents a separate embodiment of the present invention.

According to specific embodiments, the biological fluid sample is plasma or serum.

According to specific embodiments, the biological fluid sample is a plasma sample and/or a urine sample.

According to specific embodiments, the biological sample is an in-situ sample (i.e. of the cancer).

According to specific embodiments, the biological sample is cell-free.

According to other specific embodiments, the biological sample contains a cancerous cell.

According to specific embodiments, the method of the present invention comprises obtaining the biological sample prior to the determining.

The biological sample can be obtained using methods known in the art such as using a syringe with a needle, a scalpel, fine needle aspiration (FNA), catheter and the like. According to specific embodiments the biological sample is obtained by blood sampling urine collection.

According to specific embodiments, the biological sample is obtained by biopsy.

Hence, according to specific embodiments, determining the level of urea and/or pyrimidine synthesis metabolite is effected ex-vivo or in-vitro.

Determining the level of urea can be effected by any method known in the art. Conventional methods are well known in the art and are routinely used in e.g. clinical labs.

According to specific embodiments, the urea level is determined by a chemical reaction, such as but not limited to, a reaction of diacetyl with urea to form diazine, which absorbs light at 540 nm. According to other specific embodiments, the urea level is determined by an enzymatic reaction, such as but not limited to, the use urease (urea aminohydrolase, E.C. No 3.5.1.5) to generate ammonia and detection of ammonium by further reaction with GLDH, ICDH, colored chromogen or employing an ion-selective electrode.

As used herein, the phrase “pyrimidine synthesis metabolite” refers to a metabolite part of the de-novo synthesis pathway of pyrimidines including carbamoylaspartate, dihydroorotic acid (dihydroorotate), orotic acid, orotidylic acid, orotidine, orotidine monophosphate (OMP), uridine mono-phosphate (UMP), uridine di-phosphate (UDP), uridine tree-phosphate (UTP), TMP, CTP, Uracil, Tyhmidine, Cytosine.

According to specific embodiments, the pyrimidine synthesis metabolite is selected from the group consisting of Uracil, Thymidine, Orotic acid and Orotidine.

Determining the level of pyrimidine synthesis metabolite can be effected by any method known in the art, such as but not limited to LC-MS.

According to specific embodiments, the level of the pyrimidine synthesis metabolite is determined in a urine sample.

According to specific embodiments, the level of urea is determined in a blood, plasma or a serum sample.

According to a specific embodiment, the level of urea is determined in a plasma sample.

According to specific embodiments, the method of the present invention comprises determining a level of urea and a pyrimidine synthesis metabolite.

Thus, according to specific embodiments, the method of the present invention comprises determining a level of urea and a pyrimidine synthesis metabolite and wherein a ratio of the pyrimidine synthesis metabolite level to the urea level above a predetermined threshold is indicative of cancer and/or poor prognosis.

As used herein the phrase “predetermined threshold” refers to a level (typically a range) of urea and/or pyrimidine synthesis metabolite that characterizes a healthy sample. Such a level can be experimentally determined by comparing samples with normal levels of urea and/or pyrimidine synthesis metabolites (e.g., samples obtained from healthy subjects e.g., not having cancer) to samples derived from subjects diagnosed with cancer. Alternatively, such a level can be obtained from the scientific literature and from databases.

According to specific embodiments, the decrease/increase below or above a predetermined threshold is statistically significant.

According to a specific embodiment, the predetermined threshold for a pyrimidine synthesis metabolite in a urine sample is more than 0 mmoles/mol creatinine.

According to specific embodiments, the predetermined threshold is derived from a control sample.

Several control samples can be used with specific embodiments of the present invention. Typically, the control sample contains urea and/or pyrimidine synthesis metabolite in levels representative of a healthy biological sample.

Since biological characteristics depend on, amongst other things, species and age, it is preferable that the control sample is obtained from a subject of the same species, age, gender and from the same sub-population (e.g. smoker/nonsmoker).

According to specific embodiments, the control sample is from the same type as the biological sample obtained from the subject.

According to specific embodiments, the control sample is a healthy control sample.

According to specific embodiments, the control sample is a non-cancerous tissue obtained from said subject.

According to specific embodiments, the control sample is a cancerous tissue with urea level and/or pyrimidine synthesis metabolite level similar to the urea level and/or pyrimidine synthesis metabolite level in a healthy tissue of the same type.

According to specific embodiments, the control sample is obtained from the scientific literature or from a database, such as the known age matched mean value in a non-cancerous population.

According to specific embodiments, the predetermined threshold is at least 1.1 fold, at least 1.2 fold, at least 1.3 fold, at least 1.4 fold, at least 1.5 fold, at least 2 fold, at least 3 fold, at least 5 fold, at least 10 fold, or at least 20 fold as compared the level of the component in a control sample as measured using the same assay such as chromatography and mass spectrometry, enzymatic and/or chemical assay suitable for measuring expression of the compound, as further disclosed hereinabove.

According to a specific embodiment, the predetermined threshold is at least 1.1 fold compared to a control sample.

According to specific embodiments, the predetermined threshold is at least 2%, at least 5%, at least 10%, at least 20%, at least 30%, at least 40%, at least 50%, at least 60%, at least 70%, at least 80%, at least 90%, e.g., 100%, at least 200%, at least 300%, at least 400%, at least 500%, at least 600% as compared the level of the component in a control sample.

According to specific embodiments, the methods of the present invention further comprising corroborating the diagnosis and/or the prognosis using a state of the art technique.

Such methods are known in the art and depend on the cancer type and include, but not limited to, complete blood count (CBC), tumor marked tests (also known as biomarkers), imaging (such as MRI, CT scan, PET-CT, ultrasound, mammography and bone scan), endoscopy, colonoscopy, biopsy and bone marrow aspiration.

As the levels of urea and/or a pyrimidine synthesis metabolite can be used for diagnosing and/or prognosing cancer, the present invention also contemplates methods of treating and monitoring cancer treatment efficacy in subject in need thereof.

Thus, according to an aspect of the present invention, there is provided a method of monitoring efficacy of cancer therapy in a subject, the method comprising determining a level of urea and/or a pyrimidine synthesis metabolite in a biological sample of the subject undergoing or following the cancer therapy, wherein:

(i) an increase in the level of said urea; and/or

(ii) a decrease in the level of said pyrimidine synthesis metabolites; from a predetermined threshold or in comparison to said level in said subject prior to said cancer therapy, indicates efficacious cancer therapy.

According to specific embodiments, the method comprising determining said level of said urea and said pyrimidine synthesis metabolite and wherein a decrease in the ratio of said pyrimidine synthesis metabolite level to said urea level from a predetermined threshold or in comparison to said ratio in said subject prior to said cancer therapy, indicates efficacious cancer therapy.

Thus, an increase in the level of urea, a decrease in the level of a pyrimidine synthesis metabolite and/or a decrease in the ratio of the pyrimidine synthesis metabolite level to the urea level is indicative of the cancer therapy being efficient. On the other hand, if there is no change in the metabolites levels, or in case there is a decrease in the level of urea, an increase in the level of the pyrimidine synthesis metabolite or a decrease in the ratio of the pyrimidine synthesis metabolite level to the urea level, then the cancer therapy is not efficient in eliminating (e.g., killing, depleting) the cancerous cells from the treated subject and additional and/or alternative therapies (e.g., treatment regimens) may be used.

According to specific embodiments of this aspect of the present invention, the predetermined threshold is in comparison to the level in the subject prior to cancer therapy.

According to specific embodiments of this aspect of the present invention, the predetermined threshold is at least 1.1 fold, at least 1.2 fold, at least 1.3 fold, at least 1.4 fold, at least 1.5 fold, at least 2 fold, at least 3 fold, at least 5 fold, at least 10 fold, or at least 20 fold as compared the level of the component in a control sample or in the subject prior to the cancer therapy as measured using the same assay such as chromatography and mass spectrometry, enzymatic and/or chemical assay suitable for measuring expression of the compound.

According to a specific embodiment, the predetermined threshold is at least 1.1 fold as compared the level of the component in a control sample or in the subject prior to the cancer therapy.

According to specific embodiments, the predetermined threshold is at least 2%, at least 5% , at least 10%, at least 20%, at least 30%, at least 40%, at least 50%, at least 60%, at least 70%, at least 80%, at least 90%, e.g., 100%, at least 200%, at least 300%, at least 400%, at least 500%, at least 600% as compared the expression level of the component in a control sample or in the subject prior to the cancer therapy.

According to other specific embodiments of this aspect of the present invention, the pre-determined threshold can be determined in a subset of subjects with known outcome of cancer therapy.

According to another aspect of the present invention there is provided a method of treating cancer in a subject in need thereof, the method comprising:

(a) diagnosing or prognosing the subject according to the methods described herein; and herein when a

(i) level of said urea below a predetermined threshold;

(ii) level of said pyrimidine synthesis metabolite above a predetermined threshold; and/or

(iii) ratio of said pyrimidine synthesis metabolite level to said urea level above a predetermined threshold;
is indicated

(b) treating said subject with a cancer therapy.

According to another aspect of the present invention there is provided a method of treating cancer in a subject in need thereof, the method comprising:

(a) prognosing the subject according to the methods described herein; and

(b) treating said subject with a cancer therapy according to the prognosis.

According to another aspect of the present invention there is provided a method of treating cancer in a subject in need thereof, the method comprising:

(a) diagnosing or prognosing the subject according to the methods described herein; and wherein when a

(i) level of said urea below a predetermined threshold;

(ii) level of said pyrimidine synthesis metabolite above a predetermined threshold; and/or

(iii) ratio of said pyrimidine synthesis metabolite level to said urea level above a predetermined threshold;
is indicated

(b) selecting a cancer therapy based on the level of said urea and/or pyrimidine synthesis metabolite.

According to another aspect of the present invention there is provided a method of treating cancer in a subject in need thereof; the method comprising:

(a) prognosing the subject according to the methods described herein; and

(b) selecting a cancer therapy based on the prognosis.

The term “treating” refers to inhibiting, preventing or arresting the development of a pathology (e.g. cancer) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

According to specific embodiments, the cancer therapy is selected based on the prognosis of the cancer. That is, a cancer with poor prognosis is treated with a treatment regime suitable for poor prognosis according to e.g. established protocols; while cancer with good prognosis is treated with a treatment regime suitable for good prognosis according to other e.g. established protocols.

As the teachings of the present invention disclose that prognosis of the cancer is indicated by the levels of urea and/or a pyrimidine synthesis metabolite; according to specific embodiments, the cancer therapy is selected based on the levels of the determined component.

As used herein, the phrase “cancer therapy” refers to any therapy that has an anti-tumor effect including, but not limited to, anti-cancer drugs, radiation therapy, cell transplantation and surgery.

The anti-cancer drugs used with specific embodiments of the present invention include chemotherapy, small molecules, biological drugs, hormonal therapy, antibodies and targeted therapy.

According to specific embodiments, the cancer therapy is selected from the group consisting of radiation therapy, chemotherapy and immunotherapy.

Anti-cancer drugs that can be used with specific embodiments of the invention include, but are not limited to: Acivicin; Aclarubicin; Acodazole Hydrochloride; Acronine; Adriamycin; Adozelesin; Aldesleukin; Altretamine; Anibomycin; Ametantrone Acetate; Aminoglutethimide; Arnsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexonnaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enprornate; Epipropidine; Epirubicin Hydrochloride; Erbulozole; Esorubicin Hydrochloride; Estramustine; Estramustine Phosphate Sodium; Etanidazo; Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin. Sodium; Gemcitabine; Gemcitabine Hydrochloride; Hydroxyurea; Idarubicin Hydrochloride; ifosfamide; ilmofosine; Interferon Alfa-2a; Interferon Alfa-211; interferon Alfa-n1; Interferon Alfa-n3; Interferon Beta-Ia; Interferon Gamma-Ib; Iproplatin; Irinotecan Hydrochloride; Larireotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Sulofenur; Talisomycin; Taxol; Tecogalan Sodium; Tegafur; Teloxantrone Hydrochloride; Temoporfin; Teniposide; Teroxirone; Testolactone; Thiamiprine; Thioguanine; Thiotepa; Tiazofuirin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride. Additional antineoplastic agents include those disclosed in Chapter 52, Antineoplastic Agents (Paul Calabresi and Bruce A. Chabner), and the introduction thereto, 1202-1263, of Goodman and Gilman's “The Pharmacological Basis of Therapeutics”, Eighth Edition, 1990, McGraw-Hill, Inc. (Health Professions Division).

Non-limiting examples for anti-cancer approved drugs include: abarelix, aldesleukin, aldesleukin, alemtuzumab, alitretinoin, allopurinol, altretamine, amifostine, anastrozole, arsenic trioxide, asparaginase, azacitidine, AZD9291, AZD4547, AZD2281, bevacuzimab, bexarotene, bleomycin, bortezomib, busulfan, calusterone, capecitahine, carboplatin, carmustine, celecoxib, cetuximab, cisplatin, cladribine, clofarabine, cyclophosphamide, cytarabine, dabrafenib, dacarbazine, dactinomycin, actinomycin D, Darhepoetin alfa, Darbepoetin alfa, daunorubicin liposomal, daunorubicin, decitabine, Denileukin diftitox, dexrazoxane, dexrazoxane, docetaxel, doxorubicin, dromostanolone propionate, Elliott's B Solution, epirubicin, Epoetin alfa, eflotinib, estramustine, etoposide, exemestane, Filgrastim, floxuridine, fludarabine, fluorouracil 5-FU, fulvestrant, gefitinib, gemcitabine, gemtuzumab ozogamicin, goserelin acetate, histrelin acetate, hydroxyurea, ibritumomab Tiuxetan, idarubicin, ifosfamide, imatinib mesylate, interferon alfa 2a, Interferon alfa-2b, irinotecan, lenalidomide, letrozole, leucovorin, Leuprolide Acetate, levamisole, lomustine, CCNU, ineclorethamine, nitrogen mustard, megestrol acetate, melphalan, L-PAM, mercaptopurine 6-MP, mesna, methotrexate, mitomycin C, mitotane, mitoxantrone, nandrolone phenpropionate, nelarabine, Nofetumomab, Oprelvekin, Oprelvekin, oxaliplatin, paclitaxel, palbociclib palifermin, pamidronate, pegademase, pegaspargase, Pegfilgrastim, pemetrexed disodium, pentostatin, pipobroman, plicamycin mithramycin, porfimer sodium, procarbazine, quinacrine, Rasburicase, Rituximab, sargramostim, sorafenib, streptozocin, sunitinib maleate, tarnoxifen, teniozoloniide, teniposide VM-26, testolactone, thioguanine 6-TG, thiotepa, thiotepa, topotecan, toremifene, Tositumomab, Trametinib, Trastuzumab, tretinoin ATRA, Uracil Mustard, valrubicin, vinblastine, vinorelbine, zoledronate and zoledronic acid.

According to specific embodiments, the anti-cancer drug is selected from the group consisting of Gefitinib, Lapatinib, Afatinib, BGJ398, CH5183284, Linsitinib, PHA665752, Crizotinib, Sunitinib, Pazopanib, Imatinib, Ruxolitinib, Dasatinib, BEZ235, Pictilisib, Everolimus, MK-2206, Trametinib/AZD6244, Vemurafinib/Dabrafenib, CCT196969/CCT241161, Barasertib, VX-680, Nutlin3, Palbociclib, BI 2536, Bardoxolone, Vorinostat, Navitoclax (ABT263), Bortezomib, Vismodegib, Olaparib (AZD2281), Simvastatin, 5-Fluorouricil, Fluorouricil, Irinotecan, Epirubicin, Cisplatin and Oxaliplatin.

As the present invention discloses that cancer is associated with a shift from the UC to pyrimidine synthesis in the cancerous cells and decreased levels of urea and increased levels of pyrimidine synthesis metabolites in biological samples of the subject, the present inventors contemplate that cancers diagnosed, prognosed and/or monitored according to some embodiments of the present invention are more susceptible to treatment with agents targeting components associated with these pathways.

Thus, according to specific embodiments, the cancer therapy is selected from the group consisting of L-arginine depletion, glutamine depletion, pyrimidine analogs, thymidylate synthase inhibitor and mammalian target of Rapamycin (mTOR) inhibitor.

Non-limiting examples of L-arginine depletion agents which can he used with specific embodiments of the present invention include arginine deiminase (ADI) polypeptide, arginase I polypeptide, arginase II polypeptude, arginine decarboxylase polypeptide and arginine kinase polypeptide. A pegylated form of the indicated enzymes can also be used, according to specific embodiments, such as ADI-TEG 20 is a formulation of ADI with polyethylene glycol (PEG) having an average molecular weight of 20 kilodaltons (PEG 20) and a pegylated form of the catabolic enzyme arginase I (peg-Are, such as disclosed in Fletcher M et al., (2015) Cancer Res. 75(2):275-83). According to other specific embodiments, a cobalt-containing arginase polypeptide such as described in WO2010/051533 can be used.

Glutamine depletion agents that can be used with specific embodiments of the invention can act on intracellular and/or extracellular glutamine, e.g., on the glutamine present in the cytosol and/or the mitochondria, and/or on the glutamine present in the peripheral blood. Non-limiting examples of glutamine depleting agents include, inhibitors of eutamate-oxaloacetate-transaminase (GOT), carbamoyl-phosphate synthase, glutamine-pyruvate transaminase, glutamine-tRNA ligase, glutaminase, D-glutaminase, glutamine N-acyltransferase, glutaminase-asparaginase Aniinooxyacetate (AOA, an inhibitor of glutamate-dependent transaminase), phenylbutyTate and phenylacetate.

Non-limiting examples of pyrimidine analogs which can be used with specific embodiments of the invention include arabinosylcytosine, gemcitabine and decitabine.

Non-limiting examples of thymidilate synthase inhibitor that can be used according to specific embodiments of the present invention include fluorouracil (5-FU), capecitabine (an oral 5-FU pro-drug) and pemetrexed.

Another cancer therapy that can be used according to specific embodiments of the present invention include inhibitors of the mammalian target of Rapamycin (mTOR) pathway. Non-limiting Examples of mTOR inhibitors include Rapamycin and rapalogs [rapamycin derivatives e.g. temsirolimus (CCI-779), everolimus (RAD001), and ridaforolimus (AP-23573), deforolimus (AP23573), everolimus (RAD001), and temsirolimus (CCI-779)].

According to specific embodiments, the cancer therapy comprises an immune modulation agent.

Immune modulating agents are typically targeting an immune-check point protein.

As used herein the term “immune-check point protein” refers to an antigen independent protein that modulates an immune cell response (i.e. activation or function). Immune-check point proteins can be either co-stimulatory proteins [i.e. positively regulating an immune cell activation or function by transmitting a co-stimulatory secondary signal resulting in activation of an immune cell] or inhibitory proteins (i.e. negatively regulating an immune cell activation or function by transmitting an inhibitory signal resulting in suppressing activity of an immune cell). Numerous check-point proteins are known in the art and include, but not limited to, PD1, PDL-1, B7H2, B7H3, B7H4, BTLA-4, HVEM, CTLA-4, CD80, CD86, LAG-3, TIM-3, KIR, IDO, CD19, OX40, OX40L, 4-1BB (CD137), 4-1BBL, CD27, CD70, CD40, CD40L, GITR, CD28, ICOS (CD278), ICOSL, VISTA and adenosine A2a receptor.

According to specific embodiments, the immune modulating agent is a PD1 antagonist, such as, but not limited to an anti-PD1 antibody.

PD1 (Programmed Death 1), gene symbol PDCD1, is also known as CD279. According to a specific embodiment, the Pat protein refers to the human protein, such as provided in the following GenBank Number NP_005009.

Anti-PD1 antibodies suitable for use in the invention can be generated using methods well known in the art. Alternatively, art recognized anti-PD1 antibodies can be used. Examples of anti-PD1 antibodies are disclosed for example in Topalian, et al. NEJM 2012, U.S. Pat. Nos. 7,488,802; 8,008,449; 8,609,089; 6,808,710; 7,521,051; and 8168757, US Patent Application Publication Nos. US20140227262; US20100151492; US20060210567; and US20060034826 and International Patent Application Publication Nos. WO2008156712; WO2010089411; WO2010036959; WO2011159877; WO2013/019906; WO 2014159562; WO 2011109789; WO 01/14557; WO 2004/004771; and WO 2004/056875, which are hereby incorporated by reference in their entirety.

Specific anti-PD1 antibodies that can be used according to some embodiments of the present invention include, but are not limited to, Nivolumab (also known as MDX1106, BMS-936558, ONO-4538, marketed by BMY as Opdivo); Pembrolizumab (also known as MK-3475, Keytruda, SCH 900475, produced by Merck); Pidilizumab (also known as CT-011, hBAT, hBAT-1, produced by CureTech); AMP-514 (also known as N/I.EDE-0680, produced by AZY and MedImmune); and Humanized antibodies h409A11, h409A16 and h409A17, which are described in PCT Patent Application No. WO2008/156712.

According to specific embodiments, the immune modulating agent is a CTLA4 antagonist, such as, but not limited to an anti-CTLA4 antibody.

CTLA4 (cytotoxic T-lymphocyte-associated protein 4), is also known as CD152. According to a specific embodiment the CTLA-4 protein refers to the human protein, such as provided in the following GenBank Number NP_001032720.

Anti-CTLA4 antibodies suitable for use in the invention can be generated using methods well known in the art. Alternatively, art recognized anti-CTLA4 antibodies can be used. Examples of anti-CTLA4 antibodies are disclosed for example in Hurwitz et al. (1998) Proc. Natl. Acad. Sci. USA 95(17): 10067-10071; Camacho et al. (2004) J. Clin. Oncology 22(145): Abstract No. 2505 (antibody CP-675206); and Mokyr et al. (1998) Cancer Res. 58:5301-5304; U.S. Pat. Nos. 5,811,097; 5,855,887; 6,051,227; 6,207,157; 6,207,156; 6,682,736; 6,984,720; 5,977,318; 7,109,003; 7,132,281; 8,993,524 and 7,605,238, US Patent Application Publication Nos. 09/644,668; 2005/0201994; 2002/086014, International Application Publication Nos. WO2014066834; WO 01/14424 and WO 00/37504; WO2002/0039581; WO 98/42752; WO 00/37504; WO 2004/035607; and WO 01/14424, and European Patent No. EP1212422B1, which are hereby incorporated by reference in their entirety.

Specific anti-CTLA4 antibodies that can be used according to some embodiments of the present invention include, but are not limited to Ipilimumab (also known as 10D1, MDX-D010), marketed by BMS as Yervoy™; and Tremelimumab, (ticilimumab, CP-675,206, produced by MedImmune and Pfizer).

As the present invention discloses that the a shift from the UC to pyrimidine synthesis and the pyrimidine-rich transversion mutational bias enhance the response to immune-modulation therapy independently of mutational load both in mouse models and in patient correlative studies, the present inventors contemplate that cancers diagnosed, prognosed and/or monitored according to some embodiments of the present invention are more susceptible to treatment with immune-modulation therapy in combination with agents that specifically promote pyrimidines to purines nucleotide imbalance.

Thus, according to specific embodiments, the cancer therapy comprises an agent which induces a pyrimidines to purines nucleotide imbalance.

According to a specific embodiment, the cancer therapy comprises an immune modulation agent and an agent which induces a pyrimidines to purines nucleotide imbalance.

As used herein the term “induces a pyrimidines to purines nucleotide imbalance” refers to an increase in the ratio of pyrimidines to purines in a cell in the presence of the agent as compared to same in the absence of the agent, which may be manifested in e.g. increased levels of pyrimidines, decreased levels of purines and/or increased level of purine to pyrimidine transversion mutations.

According to specific embodiments, the increase is at least 1.1 fold, at least 1.2 fold, at least 1.3 fold, at least 1.4 fold, at least 1.5 fold, at least 2 fold, at least 3 fold, at least 5 fold, at least 10 fold, or at least 20 fold in the ratio of pyrimidines to purines in a cell in the presence of the agent as compared to same in the absence of the agent, which may be determined by e.g. chromatography and mass spectrometry (e.g. LC-MS), whole genome sequencing, DNA sequencing and/or RNA sequencing.

According to specific embodiments, the predetermined threshold is at least 2%, at least 5% , at least 10%, at least 20%, at least 30%, at least 40%, at least 50%, at least 60%, at least 70%, at least 80%, at least 90%, e.g., 100%, at least 200%, at least 300%, at least 400%, at least 500%, at least 600% in the ratio of pyrimidines to purines in a cell in the presence of the agent as compared to same in the absence of the agent.

According to specific embodiments, the agent which induces a pyrimidines to purines nucleotide imbalance comprises an anti-folate agent.

Anti-folate agents which can be used with specific embodiments of the invention are known in the art and include, but not limited to, methotrexate, pemetrexed, proguanil, pyrimethamine, trimethoprim, aminopterin, trimetrexate, edatrexate, piritrexim, ZD1694, lometrexol, AG337, LY231514 and 1843U89.

According to specific embodiments, the anti-folate agent comprises methotrexate.

As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention.

Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed, (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and. Higgins S. J., eds. (1984); “Animal Cell Culture” Freshney, ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein.

Other general references are provided throughout this document. The procedures therein are believed to be well known in the art and are provided for the convenience of the reader. All the information contained therein is incorporated herein by reference.

### MATERIALS AND METHODS

Determination of the urea cycle genes dysregulation score (UCD-score)—The UCD-score is a weighted sum of rank-normalized expression of the 6 urea cycle (UC) genes—ASL, ASS1, CPS1, OTC, SLC25A13 and SLC25A15; wherein:

+1 was assigned as weight for the genes CPS1 and SLC25A13;

−1 was assigned as weight for the genes ASL, ASS1, OTC and SLC25A15.

Evaluation of UC genes expression in patient samples from “The Cancer Genome Atlas (TCGA)”—TCGA gene expression profiles of 5,645 patients samples (comprising 629 normal samples) encompassing samples from 13 cancer types and a substantial number of healthy control samples (>10 for each cancer type) were downloaded from the Broad Firehose resources on Jan. 28, 2016, doi:10.7908/C11G0KM9).

Following, expression levels of 6 genes involved in the UC (i.e. ASL, ASS', CPS1, OTC, SLC25A13 and SLC25A15) in the cancer patients were compared to their expression in the healthy controls using the Student's T-test and the UCD-score was calculated. Components with significant fold changes in specific tumor types are presented in FIG. 3A. The differences remain significant vs. random shuffling of cancer/normal labels in each cancer type (P<1E-6) and random choice of sets of metabolites of similar size (P<2.67E-3). Based on the UCD-score, tumor samples were divided equally into 5 bins; and CAD expression (rank-normalized across the samples in each cancer type) was compared across these bins using a Wilcoxon rank sum test (FIG. 3D).

TCGA DNA mutation analysis—TCGA mutation profiles of 7,462 tumor samples encompassing 18 cancer types were downloaded from cbioportal¹⁸ on Feb. 1, 2017. The data from cbioportal does not include healthy control samples but integrates the mutation analysis from different TCGA centers to avoid center specific bias in mutation calls. Samples with less than 5 mutation events were excluded from further analysis.

For analyses that involved comparison within each cancer type, the 13 cancer types that had sufficient sample size (N>150), which results in 983,404 single point mutation events (including 745,712 non-synonymous mutations) in 4963 samples, were used. The fraction of transeversions from purines (R) to pyrimidines (Y), denoted herein as f(R->Y), was determined per each sample and was defined as the fraction of R->Y point mutations over all point mutations occurring in a given sample. In order to study the downstream effects of purine to pyrimidine mutations the transversion rates were quantified based on the coding (sense) strand (i.e. the TCGA mutation data was converted to its complementary sequences in genes transcribed from the (-)-strand of the genomic DNA). The fraction of transversions from pyrimidines to purines, denoted herein as f(Y->R), was determined and defined in an analogous manner Following, the association between UC dysregulation and R->Y transverse mutations was analyzed using four different approaches:

1. The R->Y mutation rates in UC dysregulated samples (top 30% of UCD-score, denoted herein as UC-dys) was compared to the R->N⁷ mutation rates in UC intact samples (bottom 30% of UCD-score, denoted herein as UC-WT) at the pancancer level and in each cancer type individually (FIG. 6B) using a Wilcoxon rank sum test.

2. The difference between R->Y and Y->R mutation rates in UC dysregulated samples (top 30%) was compared to the difference between R->Y and Y->R mutation rates in UC intact samples (bottom 30%) at the pancancer level and in each cancer type individually (FIG. 7B) using a Wilcoxon rank sum test.

3. The correlation across cancer types between median UCD-score and median pyrimidine mutation bias (f(R->Y)−f(Y->R)) of each cancer type was analyzed using Spearman correlation analysis (FIG. 7C).

4. Assessing whether purine to pyrimidine mutations associated with UC dysregulation was positively selected, based on the premise that a greater rate of non-synonymous mutations relative to synonymous mutations is indicative of positive selection. To this end, the normalized fraction of nonsynonymous purine to pyrimidine mutations in UC dysregulated vs. UC intact samples was determined (FIG. 7C). Specifically, the selective advantage (S) of R->Y mutation was estimated by the formula:

$$S = \frac{{N}_{R \to Y}}{{N}_{R \to Y} + {S}_{R \to Y}} / \frac{{N}_{all}}{{N}_{all} + {S}_{all}} , \qquad ( 1 )$$

Where N_{R->Y} denotes nonsynonymous mutation level of purine to pyrimidine transversions;
S_{R->Y} denotes synonymous mutation level of purine to pyrimidine transversions;
Nₐₗₗ denotes nonsynonymous mutation level of all mutation events; and
Sₐₗₗ denotes synonymous mutation level of all mutation events.

For this specific analysis, additional TCGA samples which had less than 5 mutation events either for synonymous or nonsynonymous mutations were filtered out, leading to 4817 samples in 13 cancer types.

Patient survival analysis—Kaplan Meier analysis and Cox proportional hazard model were performed to identify the association of UM-score with patient survival (according to the TCGA cBioportal data described above). The survival of patients with high-UCD score (top 30) and low-UCD score (bottom 30%) were compared using the logrank test¹⁹, and the effect size was quantified by the difference in the area under the curves (ΔAUC). To control for potential confounders, a Cox regression analysis was performed, while controlling for patients' age, sex, race, and cancer types, as follows:

h_{S}(t, patient)˜h_{OS}(t)exp(β_{UCD}*UCD+β_{age}*age) (2)

Where s is an indicator variable over all possible combinations of patients' stratifications based on race, sex and cancer type;
hₛ is the hazard function (defined as the risk of death of patients per time unit); and hₒₛ(t) is the baseline-hazard function at time t of the s^{th} stratification.

The model contains two covariates: (i) UCP: UCD-score based on the urea cycle deregulation signatures, and (ii) age: age of the patient. The βs are the regression coefficients of the covariates, which quantify the effect of covariates survival, determined by standard likelihood maximization of the model¹⁹ . The results of this analysis are presented in (FIG. 3E).

Detection of somatic mutations in DNA and RNA—To capture variants in the coding region, exome-seq data of 18 individual cancer and matched normal cohorts was downloaded from TCCA portal. For each BAM file of normal and cancer variants were called using the GATK (V. 3.6) ‘HaplotypeCaller’^{20,21} utility with same hg38 assembly that the TCGA used for exome-seq mapping and applying ‘-ERC GVCF’ mode to produce a comprehensive record of genotype likelihoods for every position in the genome regardless of whether a variant was detected at that site or not.

The purpose of using the GVCF mode was to capture confidence score for every site represented in a paired normal and cancer cohort for detecting somatic mutation in cancer. Following, the paired GVCFs from each paired cohorts was combined using GATK's ‘GenotypeGVCFs’ utility yielding genotype likelihood scores for every variant in cancer and the paired normal sample. In the next step GATK's ‘VariantRecalibrator’ utility using dbSNP VCF (v146:

ftp://ftp(dot)ncbi(dot)nlm(dot)nih(dot)gov/snp/organisms/human_9606_b146_GRCh38p 2/VCF) file was used by selecting annotation criteria of QD;MQ;MQRankSum;ReadPosRankSum;FS;SOR, followed by GATK's ‘ApplyRecalibration’ utility with ‘SNP’ mode. Using GATK's ‘VariantFiltration’ utility the variants with VQSLOD>=4.0 were selected. Finally, somatic mutations were defined as the loci whose genotype [1/1, 0/1, or 0/0 with ‘PL’ (Phred-scaled likelihood of the genotype) score=0, i.e., highest confidence] in cancer was distinct from that in the paired normal. The final somatic mutations were mapped on an exonic site of a transcript by ‘bcftools’ tool (V.1.3)²¹ using BED file of coding region in hg38 assembly.

To capture variants in RNA, BAM files of RNA-Seq data was downloaded for the same normal and cancer cohorts as described above. GATK's ‘SplitNCigarReads’ utility was used to split the reads into exon segments and hard-clipped to any sequence overhanging into the intronic regions. Following, GATK's ‘HaplotypeCaller’ utility was used with the same hg38 assembly that the TCGA used for RNA-Seq mapping.

To reduce false positive and false negative calls the ‘dontUseSofiClippedBases’ argument with the ‘HaplotypeCaller’ with minimum phred-scaled confidence threshold was used for calling variants set to be 20. Following, the variants were filtered using ‘VariantFiltration’ utility based on Fisher Strand values (FS>30) and Qual By Depth values (QD<2.0). Each of the output VCF files was used for annotation of coding regions on the transcripts to which the variants were mapped by using ‘bcftools’ with BED file of coding region in hg38 assembly. Based on this data, the overall R->Y mutation bias, f(R->Y)-f(Y->R) was compared between UC dysregulated vs. UC intact samples using Wilcoxon rank sum test.

Detection of somatic mutations in the proteome—To map the DNA variants to protein sequence, peptide spectrum (PSM) data was downloaded for 42 breast cancer samples, out of which only 4 samples overlapped with the samples analyzed for DNA mutations calls above. For each transcript in the somatic variant VCF file, complete coding sequence of RNA was constructed using the GATK's ‘FastaAlternateReferenceMaker’ utility. On this variant incorporated coding sequence, a codon affected by this variant site was captured and in-silico translated into an amino acid. A change was considered as a ‘non-synonymous’ change if the translated amino acid differed from the reference amino acid; and otherwise ‘synonymous’. Based on this data, the overall R->Y mutation-mapped amino acid changes we compared between UC dysregulated vs. UC intact samples using the Wilcoxon rank sum test.

Genome-scale metabolic network modeling—genome-scale metabolic modeling was used to study the stoichiometric balance of nitrogen metabolism between urea production and pyrimidine synthesis. For a metabolic network with m metabolites and n reactions, the stoichiometric constraints can be represented by a stoichiometric matrix S, as follows:

$${\sum }_{j} {S}_{ij} {v}_{j} = 0 , \qquad ( 3 )$$

where the entry Sᵢⱼ represents the stoichiometric coefficients of metabolite i in reaction j, and vⱼ stands for the metabolic flux vector for all reactions in the model. The model assumes steady metabolic state, as represented in equation (3) above, constraining the production rate of each metabolite to be equal to its consumption rate. In addition to the mass balance, a constraint-based model limits the space of possible fluxes in the metabolic network's reactions through a set of (in)equalities imposed by thermodynamic constraints, substrate availability and the maximum reaction rates supported by the catalyzing enzymes and transporting proteins, as follows:

αⱼ≤νⱼ≤βⱼ, (4)

where αⱼ and βⱼ defines the lower and upper bounds of the metabolic fluxes for different types of metabolic fluxes. (i) The exchange fluxes model the metabolite exchange of a cell with the surrounding environment via transport reactions, enabling a pre-defined set of metabolites to be either taken up or secreted from the growth media. (ii) Enzymatic directionality and flux capacity constraints define lower and upper bounds on the fluxes as represented in equation (4) above. The human metabolic network model²⁴ was used with biomass function introduced in Folger et al²⁵ under the Roswell Park Memorial Institute Medium (RPMI)-1640.

To study the metabolic alterations occurring in UE dysregulated cancer cells (having increased growth and biomass production rates, and increase CAD activity versus healthy cells), a flux-balance-based analysis²³ was performed. The maximal production rate of urea was computed while gradually increasing the demand constraints for biomass production rates and the flux via the three enzymatic reactions of CAD Carbamoyl-phosphate synthetase 2 (CPS2), Aspartate transcarbamylase (ATC) and Dihydroorotase—up to their maximal feasible values in the model (FIG. 5A, right). In addition, the nitrogen utilization in each of the conditions sampled in the procedure above was computed, by subtracting the total amount of nitrogen excreted from the amount of nitrogen uptake, while taking into account the nitrogen's stoichiometry in all nitrogen-containing metabolites (FIG. 5A, left).

Joint transcriptomic and inetabolomic analysis of tumor samples—Recently published data of joint transcriptomic and metabolomic measurements across 58 breast cancer (BC) tumors vs. healthy controls²³ and 29 hepatocellular carcinoma (HCC) samples vs. healthy controls ²⁴ was analyzed, to further study the association between UC dysregulation and metabolites levels in clinical samples. For each sample, a score denoting the ratio of pyrimidine to purine metabolite levels in the given sample was computed. Following, the samples were divided into two groups based on their UCD-scores and the two groups were compared using Wilcoxon rank-sum, in each of the two cancer types (FIG. 5C).

Patient samples—Plasma urea levels measurements were taken from Hemato-Oncology patients' medical record excluding patients' identifiers and with approval by the ethic committee (TLV 0016-17). Prostate specimens were obtained upon informed consent and with evaluation and approval from the corresponding ethics committee (CEIC code OHEUN11-12 and OHEUN14-14). Blood samples were taken from patients diagnosed with benign prostate hyperplasia (BPH) with normal PSA levels or with prostate adenocarcinoma (PCa); and a scheduled surgery as anticancer treatment (PCa) or to alleviate disease-related symptoms (BPH) served as an inclusion criteria. The biopsy-based diagnosis was corroborated in the surgical piece. The blood was collected following overnight fasting and prior to surgery. Plasma was extracted from the blood samples and analysed for urea concentration, following standard clinical procedures. Following urea concentration analysis, outliers were removed using the ROUT method (Q=1%).

Cell and cell cultures—Patients fibroblast studies were performed anonymously on cells devoid of all patient identifiers. Punch biopsies were taken from UC deficient patients to generate fibroblast cell line. HepG2 cell line was purchased from ATTC. OTC and CPS1 deficient cell lines as well as control fibroblasts were purchased from Coriell Institute for Medical Research (GM06902; GM12604). Cells were cultured using standard procedures in a 37° C. humidified incubator with 5% CO₂ in Dulbecco's Modified Eagle's Medium (DMEM, sigma-aldrich) supplemented with 10-20% heat-inactivated fetal bovine serum, 10% pen-strep and 2 mM glutamine. All cells were tested routinely for Mycoplasma using Mycoplasma EZ-PCR test kit (#20-700-20, Biological Industries, Kibbutz Beit Ha'emek). Crystal Violet Staining—Cells were seeded in 12-wells plates at 75,000-150,000 cells well in triplicates. Time 0 was determined as the time the cells adhered to the culture plate, which was about 10 hours following seeding. For each time point, cells were washed with PI3S X1 and fixed in 4% PFA (in PBS). Following, cells were stained with 0.5% Crystal Violet (Catalog number C0775, Sigma-Aldrich) for 20 minutes (1 ml per well) and washed with water. The cells were then incubated with 10% acetic acid for 20 minutes with shaking. The extract was diluted 1:1-1:4 in water and absorbance was measured for each time point at 595 nm every 24 hours.

Immunohistochemistry—Four micrometer paraffin embedded tissue sections were deparaffinized and rehydrated. Endogenous peroxidase was blocked with three percent H₂O₂ in methanol. For ASL, ASS1 and ORNT1 (SLC25A15) staining, antigen retrieval was performed in citric acid (pH 6), for 10 minutes, using a low boiling program in the microwave to break protein cross-links and unmask antigens. Following, the sections were pre-incubated with 20% normal horse serum and 0.2% Triton X-100 for 1 hour at RT, biotin block via. Avidin/Biotin Blocking (SP-2001, Vector Laboratories, Ca, USA). The blocked sections were incubated overnight at room temperature followed by 48 hours at 4° C. with the following primary antibodies: ASL (1:50, Abcam, ab97370, CA, USA); ASS1 (1:50, Abcam, ab124465, CA, USA), ORNT1 (1:200, NBP2-20387, novas biologicals, CO, USA), OTC (1:3-1:200, HPA000570, Sigma-aldrich). All antibodies were diluted in PBS containing 2% normal horse serum and 0.2% Triton. Following, the sections were washed three times with PBS and incubated with secondary biotinylated IgG antibody at for 1.5 hour at room temperature, washed three times in PBS and incubated with avidin-biotin Complex (Elite-ABC kit, Vector Lab, CA, USA) for additional 90 minutes at room temperature, followed by DAB (Sigma) reaction. Stained sections were examined and photographed by a bright field microscope (E600, Tokyo, Japan) equipped with Plan Fluor objectives (10×) connected to a CCd camera (DS-Fi2, Nikon). Digital images were collected and analyzed using Image Pro+ software. Images were assembled using Adobe Photoshop (Adobe Systems, San Jose, Calif.).

Viral infection—Primary fibroblasts were infected with HCMV and harvested at different times points following infection for ribosome footprints (deep sequencing of ribosome-protected mRNA fragments) as previously described²⁵. Briefly, human foreskin fibroblasts (HFF) were infected with the Merlin HCMV strain and the cells were harvested at 5, 12, 24 and 72 hours post infection. Cells were pre-treated with Cylcoheximide and ribosome protected fragments were then generated and sequenced. Bowtie v0.12.7 (allowing up to 2 mismatches) was used to perform the alignments. Reads with unique alignments were used to compute footprints densities in units of reads per kilobase per million (RPKM).

Metabolomics analysis—HepG2 cell lines were seeded at 3-5×10⁶ cells per 10 cm plate and incubated with 4 mM. L-glutamine (α-15N, 98%, Cambridge Isotope Laboratories) for 24 hours. Subsequently, cells were washed with ice-cold saline, lysed with a mixture of 50% methanol in water added with 2 μg/ml ribitol as an internal standard and quickly scraped followed by three freeze-thaw cycles in liquid nitrogen. Following, the sample was centrifuged in a 4° C. cooled centrifuge and the supernatant was collected for consequent GC-MS analysis. The pellets were dried under air flow at 42° C. using a Techne Dry-Block Heater with sample concentrator (Bibby Scientific) and the dried samples were treated with 40 μl of a methoxyamine hydrochloride solution (20 mg ml-1 in pyridine) for 90 minutes while shaking at 37° C. followed by incubation with 70 μl N,O-bis (trimethylsilyl) trifluoroacetamide (Sigma) for additional 30 minutes at 37° C.

Isotopic labeling—Hepatocellular and ovarian carcinoma cells were seeded in 10 cm plates and once cell confluency reached 80% cells were incubated with 4 mM L-GLUTAMINE, (ALPHA-15N, 98%, Cambridge Isotope Laboratories, Inc.) for 24 hours.

GC-MS analysis—GC-MS analysis used a gas chromatograph (7820AN, Agilent Technologies) interfaced with a mass spectrometer (5975 Agilent Technologies). An HP-5 ms capillary column 30m×250 μm×0.25 μm (19091S-433, Agilent Technologies) was used. Helium carrier gas was maintained at a constant flow rate of 1.0 ml min−1. The GC column temperature was programmed from 70 to 150° C. via a ramp of 4° C. min⁻¹, 250-215° C. via a ramp of 9° C. min⁻¹, 215-300° C. via a ramp of 25° C. min⁻¹ and maintained at 300° C. for additional 5 minutes. The MS was effected by electron impact ionization and operated in full-scan mode from m=30-500. The inlet and MS transfer line temperatures were maintained at 280° C., and the ion source temperature was 250° C. Sample injection (1-3 μl) was in split less mode.

Nucleotide analysis—Materials: Ammonium acetate (Fisher. Scientific) and ammonium bicarbonate (Fluka) of LC-MS grade; Sodium salts of AMP, CMP, GMP, TMP and UMP (Sigma-Aldrich); Acetonitrile of LC grade (Merck); water with resistivity 18.2 MΩ obtained using Direct 3-Q UV system (Millipore).

Extract preparation: Samples were concentrated in speedvac to eliminate methanol, and then lyophilized to dryness, re-suspended in 200 μl of water and purified on polymeric weak anion columns [Strata-XL-AW 100 μm (30 mg ml⁻¹, Phenomenex)] as follows: each column was conditioned by passing 1 ml of methanol followed by 1 ml of formic acid/methanol/water (2/25/73) and equilibrated with 1 ml of water. The samples were loaded, and each column was washed with 1 ml of water and 1 ml of 50% methanol. The purified samples were eluted with 1 ml of ammonia/methanol/water (2/25/73) followed by 1 ml of ammonia/methanol/water (2/50/50) and then collected, concentrated in speedvac to remove methanol and lyophilized. Following, the obtained residues were re-dissolved in 100 μl of water and centrifuged for 5 minutes at 21,000 g to remove insoluble material.

LC-MS analysis: The LC-MS/MS instrument used for analysis of nucleoside monophosphates was an Acquity I-class UPLC system (Waters) and Xevo TQ-S triple quadrupole mass spectrometer (Waters) equipped with an electrospray ion source and operated in positive ion mode. MassLynx and TargetLynx software (version 4.1, Waters) were applied for data acquisition and analysis. Chromatographic separation was done on a 100 mm×2.1 mm internal diameter, 1.8 μm UPLC HSS T3 column equipped with 50 mm×2.1 mm internal diameter, 1.8 μm UPLC HSS T3 pre-column (both Waters Acquity) with mobile phases A (10 mM ammonium acetate and 5 mM ammonium hydrocarbonate buffer, pH 7.0 adjusted with 10% acetic acid) and B (acetonitrile) at a flow rate of 0.3 ml min⁻¹ and column temperature 35° C. A gradient was used as follows: for 0-3 min the column was held at 0% B, 3-3.5 min a linear increase to 100% B, 3.5-4.0 min held at 100% B, 4.0-4.5 min back to 0% B and equilibration at 0% B for 2.5 min. Samples kept at 8° C. were automatically injected in a volume of 3 μl. For mass spectrometry, argon was used as the collision gas with a flow of 0.15 ml min⁻¹. The capillary voltage was set to 2.90 kV, source temperature 150° C., desolvation temperature 350° C., cone gas flow 150 l hr⁻¹, desolvation gas flow 650 l hr⁻¹.

Downregulation of OTC—HEPG2 Cells were infected with pLKO-based lentiviral vector with or without the human OTC short hairpin RNA (shRNA) encoding one or two separate sequences combined (RHS4533-EG5009, GE Healthcare, Dharmacon). Transduced cells were selected with 4 μg ml⁻¹ puromycin.

Virus infection—Primary fibroblasts were infected with HCMV and harvested at different time points following infection for ribosome footprints (deep sequencing of ribosome-protected mRNA fragments) as previously described (Tirosh et al,, 2015). Briefly human foreskin fibroblasts (HFF) were infected with the Merlin HCMV strain and harvested cells at 5, 12, 24 and 72 hours post infection. Cells were pre-treated with Cylcoheximide and ribosome protected fragments were then generated and sequenced. Bowtie v0.12.7 (allowing up to 2 mismatches) was used to perform the alignments. Reads with unique alignments were used to compute footprints densities in units of reads per kilobase per million (RPKM).

Cancer cells were infected with pLKO-based lentiviral vector with or without the human OTC and SLC25A15, ASS1 short hairpin RNA (shRNA) (Dharmacon). Transduced cells were selected with 2-4 μg m⁻¹ puromycin.

Transient transfection—LOX-IMVI melanoma cells were seeded in 6-well plates at 70,000 cells/well, or in 12-well plates at 100,000 cells/plate. At the following day, cells were transfected with either 700 pmol or 350 pmol siRNA siGenome SMARTpool targeted to human SLC25A13 mRNA (#M-007472-01, Dharmacon), respectively. Hepatocellular and ovarian carcinoma cells were seeded in 6-well plate at 10⁶ or 70,000 cells/well respectively, transfected with 2-3 μg of the OTC (EXa3688-LV207 GENECOPOEIA) or ORNT1 (EXu0560-LV207 GENECOPOEIA) plasmids. Transfection was effected with Lipofectamine® 2000 Reagent (#11668027, ThermoFisher Scientific), in the presence of Opti-MEM® I Reduced Serum Medium (#11058021, ThermoFisher Scientific). Four hours following transfection, medium was replaced and the experiments were performed 48-108 hours post transfection.

Over expression—LOX-IMVI melanoma cells were transduced with pLEX307-based lenti-viral vector with or without the human SLC25A13 transcript, encoding for Citrin. Transduced cells were selected with 2 μg/ml Purornycin.

In-vivo experiments—8 weeks old Balb/c or C57131, mice were injected with 4T1 breast cancer cells (in the mammary fat fad) or with CT26 colon cancer cells (sub-cutaneous). 3 weeks following injection an advanced tumor was observed and palpated. Urine was collected from mice presenting adverse tumors. Pyrimidine pathway related metabolites were assessed by LC-MS at Baylor. College of medicine. Control urine was obtained from Balb/c or C57BL mice similar in age which were not injected. Samples below 100 μl were excluded from the analysis. All animal experiments were approved by the Weizmann Institute Animal Care and Use Committee Following US National institute of Health, European Commission and the Israeli guidelines (IACUC 21131015-4).

Syngeneic mouse models—8 weeks old C57BL/6 male mice were injected sub-cutaneous in the right flank with MC38 mouse colon cancer cells infected with either an empty vector (EV) or with shASS 1. For each injection, 5×10⁵ tumor cells were suspended in 200 μl DMEM containing 5% matrigel. Following injection, on days 8, 13, 17, 20, mice were treated with 250 μg of anti PD-1 antibody (Clones 29F.1A12, RPM114, Bio Cell) or PBS (control) as control. On day 22, mice were euthanized and tumors were removed and incubated in 1 ml of PBS containing Ca2+, Mg2+(Sigma D8662) with 2.5 mg/ml Collagenase D (Roche) and 1 mg/ml DNase I (Roche). Following 20 minutes incubation at 37° C., the tumors were processed into a single cell suspension by mechanically grinding on top of wire mesh and repeated washing and filtering onto 70 μM filter (Falcon). Single cell suspensions from tumors were stained for flow cytometry analysis with CD3-FITC (clone 17A2), CD4-PE (clone GK15) and CD8a-APC (clone 53-6.7) all from Biolegend. Next, the cells were fixed using BD cytofixIcytoperm solution (BD Biosciences) and acquired on LSRII flow cytometer at the Weizmann FACS facility and analyzed with Floyd° software (Tree Star). The tumor volume was quantified by the formula, (l×w×h) π6, and normalized by their volume on day 11 when the mean tumor volume reached around 100 mm³. The response to anti-PD1 therapy (and empty vector) was quantified by the tumor volume change at time t, ΔVₜ=(Vₜ−V₀)/V₀, where Vₜ denotes the normalized tumor volume at a given time t, and V₀ denotes the tumor volume on day 11. The overall response of treated and control groups was compared by Wilcoxon ranksurn test of ΔVₜ on day 21, and the sequential tumor growth was compared using ANOVA over the whole period (where the internal tumor volume was measured on day 9, 13,17, and 19).

Western blotting—Cells were lysed in RIPA (Sigma-Aldrich) and 0.5% protease inhibitor cocktail (Calbiochem), 1% phosphatase inhibitor cocktail (P5726, sigma-aldrich). Following centrifugation, the supernatant was collected and protein content was evaluated by the Bradford assay. 100 μg from each sample under reducing conditions were loaded into each lane on a 10% SDS polyacrylamide gel and separated by electrophoresis.

Following electrophoresis, proteins were transferred to Immobilon transfer membranes (Tamar, Jerusalem, Israel). Nonspecific binding was blocked by incubation with TBST [10 mM Tris-HCl (pH 8.0), 150 mM NaCl, 0.1% Tween 20] containing 5% skim milk or BSA 3% (Sigma catalog no: A7906) for 1 hour at room temperature. Membranes were subsequently incubated with primary antibodies against: p97 (1:10,000, PA5-22257, Thermo Scientific), GAPDH (1:1000, 14010, #2118, Cell Signaling), CAD (1:1000, ab40800, abeam), phospho-CAD (Ser1859) (1:1000, #12662, Cell Signaling), ASL (1:1000, ab97370, Abeam), MAP2K1 (1:10000, MFCD00239713, Sigma-Aldrich), OTC (1:1000, ab203859, Abeam). Following, the membranes were incubated with the secondary antibodies used were: using peroxidase-conjugated AffiniPure goat anti-rabbit IgG or goat anti-mouse IgG (Jackson ImmunoResearch, West Grove, Pa.) and detected by enhanced chemiluminescence western blotting detection reagents (EZ-Gel, Biological Industries). The bands were quantified by Gel Doc™ XR+(BioRad) and analyzed by Image Lab 5.1 software (BioRad).

Predicting the success of immune checkpoint inhibitors therapy—Three different melanoma ICT datasets (Van Allen et al., 2015, Hugo et al., 2016 and Roh et al., 2017) treated with anti-CTLA4 therapy and anti-PD1 therapy were analyzed. The third dataset includes both anti-CTLA4 and anti-PD1, however the anti-PD1 arm was analyzed because it has a larger sample size. The definition of responders determined by the combination of RECIST criteria (treating complete response (CR) and partial response (PR) as responders and the progressive disease (PD) as non-responders) was followed. UCD-score between responders and non-responders were compared in two datasets (Van Allen et al., 2015 and Hugo et al., 2016) where UC enzymes are available using Wilcoxon ranksum test; the third dataset (Roh et al., 2017) has nanostring data, where not all of the expression of 6 UC genes are available. The association of CAD expression and ICD response was evaluated in an analogous manner. The predictive power of mutational load and PTMB for the success of anti-PD1 was evaluated in connection with the in vivo anti-PD1 experiment using ROC analysis in the datasets where the processed mutation data was available (Roh et al., 2017).

Production and purification of membrane HLA molecules—Cell line pellets were collected from 2×10⁸ cells. Cell pellets were homogenized through a cell strainer on ice with lysis buffer containing 0.25% sodium deoxycholate, 0.2 mM iodoacetamide, 1 mM EDTA, 1;300 Protease Inhibitors Cocktail (Sigma-Aldrich, P8340), 1 mM PMSF and 1% octyl-b-D glucopyranoside in PBS. Samples were then incubated at 4° C. for 1 hour. The lysates were cleared by centrifugation at 48,000 g for 60 minutes at 4° C., and then were passed through a pre-clearing column containing Protein-A Sepharose beads. HLA-I molecules were immunoaffinity purified from cleared lysate with the pan-HLA-I antibody (W6/32 antibody purified from HB95 hybridoma cells) covalently bound to Protein-A Sepharose beads. Affinity column was washed first with 10 column volumes of 400 mM NaCl, 20 mM Tris-HCl followed by 10 volumes of 20 mM Tris-HCl, pH 8.0. The HLA peptides and HLA molecules were then eluted with 1% trifluoracetic acid followed by separation of the peptides from the proteins by binding the eluted fraction to disposable reversed-phase C18 columns (Harvard Apparatus). Elution of the peptides was effected with 30% acetonitrile in 0.1% trifluoracetic acid (Milner et al., 2013). The eluted peptides were cleaned using C18 stage tips as described previously (Rappsilber et al., 2003).

Identification of eluted HLA peptides—The HLA peptides were dried by vacuum centrifugation, solubilized with 0.1% formic acid, and resolved on capillary reversed phase chromatography on 0.075×300 mm laser-pulled capillaries, self-packed with C18 reversed-phase 3.5 μm beads (Reprosil-C18-Aqua, Dr. Maisch GmbH, Ammerbuch-Entringen, Germany) (Ishihama et al., 2002). Chromatography was performed with the UltiMate 3000 RSLCnano-capillary UHPLC system (Thermo Fisher Scientific), which was coupled by electrospray to tandem mass spectrometry on Q-Exactive-Plus (Thermo Fisher Scientific). The HLA peptides were eluted with a linear gradient over 2 hours from 5 to 28% acetonitrile with 0.1% formic acid at a flow rate of 0.15 μl/minute. Data was acquired using a data-dependent “top 10” method, fragmenting the peptides by higher-energy collisional dissociation. Full scan MS spectra was acquired at a resolution of 70,000 at 200 m/z with a target value of 3×10⁶ ions. Ions accumulated to an AGC target value of 105 with a maximum injection time of generally 100 milliseconds. The peptide match option was set to Preferred. Normalized collision energy was set to 25% and MS/MS resolution was 17,500 at 200 m/z. Fragmented m/z values were dynamically excluded from further selection for 20 seconds. The MS data were analyzed using MaxQuant (Cox and Mann, 2008) version 1.5.3.8, with 5% false discovery rate (FDR). Peptides were searched against the UniProt human database (July 2015) and customized reference databases that contained the mutated sequences identified in the sample by WES. N-terminal acetylation (42.010565 Da) and methionine oxidation (15.994915 Da) were set as variable modifications. Enzyme specificity was set as unspecific and peptides FDR was set to 0.05. The match between runs option was enabled to allow matching of identifications across the samples belonging the same patient.

HLA typing was determined from the WES data by POLYSOLVER version 1.0 (Shukla et al., 2015); and the HLA allele to which the identified peptides match to was determined using the NetMHCpan version 4.0 (Hoof et al., 2009; Nielsen and Andreatta, 2016). The abundance of the peptides was quantified by the MS/MS intensity values, following normalization with the summed intensity of both UC-perturbed and control cell lines. The hydrophobicity of a peptide was determined by the fraction of hydrophobic amino acid in the peptide, which we termed hydrophobic score. The abundance of the peptides of top 20% hydrophobic score vs bottom 20% of hydrophobic score was compared using Wilcoxon ranksum test in UCD cell lines and control cell lines.

Peptidomics analysis—To identify the neo-antigens, nonsynonymous mutations in UCD perturbed cells to the mass-spec data from the un-perturbed and perturbed cells were mapped. The raw mass-spec data was transformed to mzML format using MSConvertGUI tool, integrated in ProteoWizard 3.0 (Chambers et al., 2012). The mzML files from cell lines, each from with/without UC perturbation conditions, were used as an input to RAId_DbS tool, with all default parameters and recommended settings for our application (Alves et al., 2007). 2 missed cleavage sites at most were allowed. For terminal group molecular weight (Da), the default 1.0078 and 17.0027 were chosen respectively for N-terminal and C-terminal attached chemical group, which accounts for the Hydrogen signal and —COOH group respectively. The default mass tolerance (Da) of 1.0 in precursor ion and 0.2 in product ion parameters were used. Finally, the “RAId_score” was used to identify peptides using P-value threshold of 0.05 (and E-value<=1). Following, the reference protein sequence database from NCBI (Refseq release 82) was used to map the peptides to protein IDs. In identifying single amino acid polymorphisms (SAPS) all amino acids were allowed for. The RAId_DbS outputs, each from the paired cell lines, were used to map the amino-acid change to non-synonymous mutations on genes, separately for R->Y and Y->R cases, reported in VCF files, using in-house python script.

Statistics—Statistical analyses were performed using one-way ANOVA, dependent and independent-samples Student's T-test or Wilcoxon rank sum test of multiple or two groups, with Dunnett's correction when required. Log-transformed data were used where differences in variance were significant and variances were correlated with means. The sample size was chosen in advance based on common practice of the described experiment and is indicated. Each experiment was conducted with biological and technical replicates and repeated at least three times unless specified otherwise. When samples were distributed non-normally, Mann-Whitney analysis was performed. Statistical tests were done using Statsoft's STATISTICA, ver. 10. All error bars represent statistical error (SER). P<0.05 was considered significant in all analyses (*P<0.05, **P<0.005, ***P<0.0005, ****P<0.0001).

### Example 1

### Association Between UC Dysregulation, CAD and Pyrimidine Synthesis

Patients with inborn deficiency in the UC components ornithine transcarbamylase (OTC), argininosuccinate lyase (ASL), argininosuccinate synthase (ASS1) or the transporter ornithine translocase (SLC25A15 or ORNT1) have increased pyrimidine-related metabolites in plasma or urine whereas patients with inborn carbamoyl phosphate synthetase I (CPSI) deficiency do not⁷⁻¹¹.

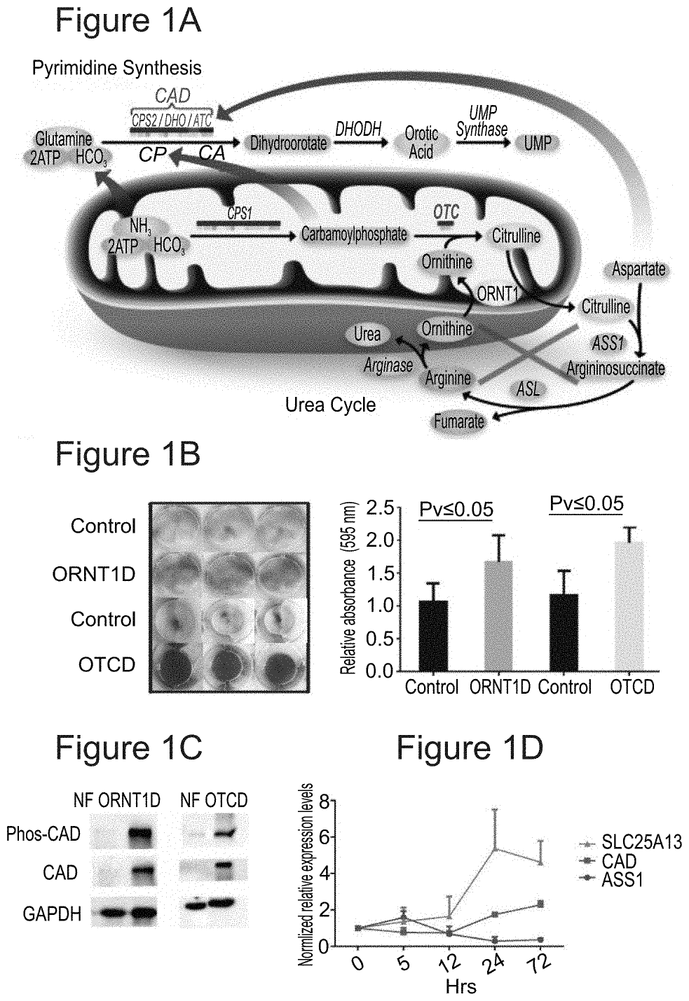

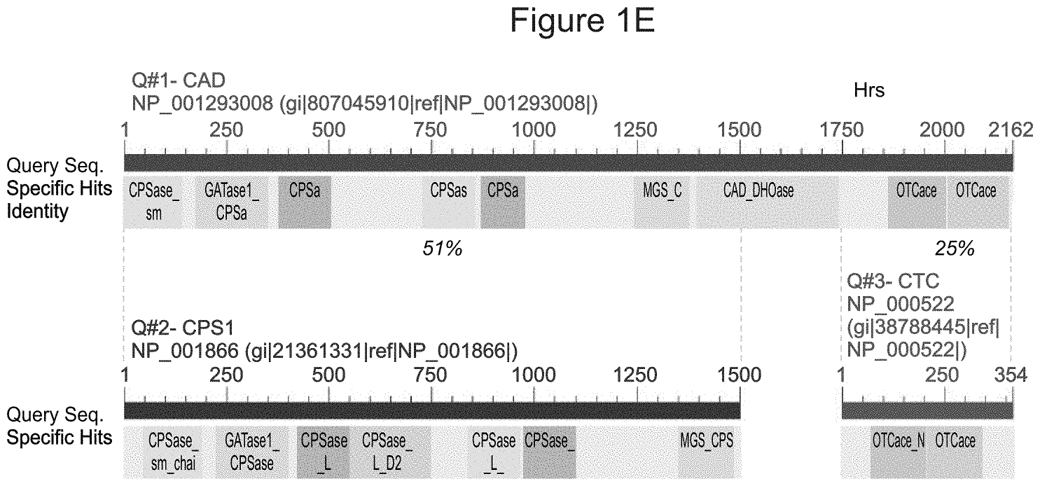

These findings raise the possibility that a block in ureagenesis in non-cancerous settings is associated with increased pyrimidine synthesis and that a specific rewiring of the UC components is required for this association (FIG. 1A). Hence, to assess the direct implications of UC dysregulation, fibroblasts from OTC deficient (OTCD) and ORNT1 deficient (ORNT1D) patients were studied. As shown in FIGS. 1 these fibroblasts were significantly more proliferative (as evident by the crystal violet stain) and exhibited elevated levels of activated CAD protein as compared to fibroblasts from healthy controls. On the contrary, fibroblasts from CPS1 deficient patients proliferated to the same extent and exhibited the same levels of activated CAD protein as fibroblasts from healthy controls (data not shown).

Additionally, cytomegalovirus infection which has been reported to cause activation of CAD and expansion of pyrimidine pools¹², leads to time dependent reduction in ASS1 expression and elevation in the UC transporter SLC25A13 levels in concordance with CAD elevation (FIG. 1D). These findings suggest a metabolic link between specific changes in UC components' expression, CAD activation, nucleotide synthesis and proliferation.

To assess the potential mechanism underlying this metabolic association an online free NCB1 protein alignment and BLAST tools were utilized, revealing high structural homology and high identity between the proximal UC enzymes, CPS1 and OTC; and between the components of the CAD-CPS2 and ATC, respectively (FIG. 1E). These findings together with the reported increased nitrogen flux through the UC over pyrimidine synthesis¹³, suggest that in multiple circumstances, diversion of metabolites from the UC enzymes to the CAD enzyme would decrease ureagenesis and substantially enhance pyrimidine synthesis and proliferation.

### Example 2

### UC Dysregulation Correlates with Cancer Prognosis

Metabolic redirection from the UC towards CAD (denoted herein as UCD) is expected from down-regulation of ASS1, ASL, OTC, or SLC25A15 (ORNT1), or from up-regulation of CPS1. or SLC25A13 (citrin). Thus, for example, as shown in FIGS. 2A-D, downregulation of ASS1 or OTC in cancer cells using shRNA resulted in increased proliferation and pyrimidine synthesis. To further substantiate this notion, in addition to downregulation of OTC in the hepatocellular carcinoma (HepG2), SLC25A15 (ORNTI) was downregulated in ovarian carcinoma (SKOV), and SLC25A13 (citrin) was overexpressed in melanoma cells (LOX IMVI). Following each specific perturbation, CAD activation was measured through its phosphorylation on serine 1859. Importantly, each of these separate perturbations led to an increase in CAD phosphorylation and enhanced cellular proliferation in vitro (FIGS. 2F-G). Furthermore, downregulation of OTC and SLC25A15 (ORNT1), resulted in increased ¹⁵N labelling of uracil from glutamine in vitro and increased tumor growth in vivo (FIG. 2H).

Taken together, UC dysregulation and the consequent flux of nitrogen towards CAD can be achieved through specific alterations in expression of different enzymes in the cycle (FIG. 1A).

To quantify the total extent of expression dysregulation in the above described 6 UC enzymes [i.e. ASS1, ASL, OTC, SLC25A15 (ORNT1), CPS1, SLC25A13 (citrin)] a UCD-score was computed. The UCD-score takes the aggregate expression of the 6 enzymes in the direction that supports metabolic redirection toward CAD. Specifically, it is a weighted sum of rank-normalized expression of the six genes across tumor samples, where ASS1, ASL, OTC, and SLC25A15 (ORNT1) take the weight of −1 and CPS1 or SLC25A13 (citrin) take the weight of +1.

By analyzing the human tumor transcriptomics data from the cancer genome atlas (TCGA) collection, the expression levels of the 6 UC genes show the alteration that supports metabolic redirection toward CAD in most TCGA tumor samples compared to their normal controls. Moreover, a majority of tumors harbour expression alterations in at least two UC components in the direction that enhances CAD activity (FIG. 3A, Table 1 below). As show in FIGS. 3B-C and 4B, UCD was also evident at the protein level. Beyond its association with CAD activity (FIGS. 3A, 3C and 4A), UCD (and the LCD-score) was associated with higher tumor grade (FIG. 4A). Importantly, both the specific changes in UC components' expression and independently, high CAD phosphorylation representing high CAD activity, were significantly associated with decreased cancer patients' survival (FIGS. 3E and 4D-E).

Taken together, UCD in cancer is a result of coordinated alterations in UC enzyme activities, where CPS1 and SLC25A13 tend to be up-regulated, while ASL, ASS1, OTC and SLC25A15 tend to be down-regulated to increase substrate supply to CAD and enhance pyrimidine synthesis (see FIG. 4A); and most importantly UCD correlates with cancer prognosis and patient's survival.

TABLE 1
Fraction of the samples of UC dysregulated and
PTMB in different cancer types.

| Tumor types | UCD samples | PTMB samples |
|---|---|---|
| LIHC | 95.5% | 79.8% |
| BLCA | 79.5% | 92.9% |
| LUSC | 72.1% | 98.8% |
| CESC | 69.6% | 83.2% |
| STAD | 66.7% | 76.5% |
| SARC | 66.1% | 65.6% |
| KIRC | 63.5% | 63.0% |
| KIRP | 61.3% | 60.9% |
| LUAD | 59.8% | 89.5% |
| HNSC | 58.5% | 81.0% |
| BRCA | 55.3% | 63.8% |
| UCEC | 42.9% | 85.7% |
| PRAD | 35.9% | 51.5% |
| LGG | 34.7% | 43.9% |
| OV | 30.8% | 67.9% |
| SKCM | 11.8% | 48.7% |

*The table lists the fraction of the TCGA samples where UCD-score is higher than the mean UCD score of corresponding healthy tissues (2nd column), and the fraction of the samples where PTMB is higher than expected (3rd column) in 15 different cancer types (1st column).

### Example 3

### Nitrogen Metabolites Can Serve as Cancer Biomarkers

Metabolic modelling of the network wide effects supports the notion that UCD would result in a diversion of nitrogenous metabolites from catabolic to anabolic processes, leading to increased synthesis of nitrogen rich metabolites, such as pyrimidines, and decreased ureagenesis (FIGS. 5A and 5F). This modelling along with the experimental results described above suggests that changes in nitrogen metabolites in cancer may be detectable in biofluids, thereby allowing non-invasive cancer monitoring. To this end, the urine nitrogenous pyrimidine metabolites of mice bearing tumors vs. disease-free animals were compared. Interestingly, increased pyrimidine synthesis related metabolites were detected in the urine of mice bearing breast or colon tumors as compared to control mice (FIG. 5B) which was accompanied with UCD (FIG. 5G). Furthermore, the analysis of purine and pyrimidine metabolites in patients with hepatocellular carcinoma and breast cancer showed a significant correlation between the UCD-score and the increase in pyrimidines (FIG. 5C).

Following, a proof-of-principle analysis in biofluids from individuals with cancer was conducted. A significantly elevated levels of pyrimidines in urine of patients with prostate cancer was found, compared to healthy controls (FIG. 5G). In addition, the medical records of cancer patients in a large medical center in Israel was surveyed and the results demonstrated that in comparison to the established age-matched mean urea values in health¹⁴, children across a broad array of cancer types have significantly decreased plasma urea levels at the day of admission (FIG. 5D). In concordance, a significant decrease in plasma urea levels we observed in 519 patients with prostate cancer when compared to 257 individuals diagnosed with benign prostate hyperplasia (FIG. 5E).

Taken together, these findings support the global dysregulation of nitrogen metabolism especially in advanced cancer that favours nitrogen utilization for pyrimidine synthesis over systemic urea disposal, resulting in identifiable nitrogen metabolites alterations in mice and cancer patients' bio-fluids and suggest monitoring these changes as cancer biomarkers.

### Example 4

### UCD is Associated with Increased Purine to Pyrimifine Transversion Mutations in Cancer

The data shows dysregulation of UC enzyme(s) in cancer resulting in increased CAD activity that leads to increased pyrimidine levels. To test this effect directly, the equilibrium between purines and pyrimidines in osteosarcoma and hepatic cancer cells upon downregulation of ASS1 and OTC, respectively, was determined As predicted, perturbed UC enzyme activity increased pyrimidine levels and significantly altered the ratio between purines and pyrimidines (FIGS. 6A and 7A). Similarly, a cellular increase in the ratio of pyrimidine to purine metabolites was also found in the other UCD induced cancer cells generated (FIG. 2F and 8).

As nucleotide imbalance has been reported to promote carcinogenesis by increasing mutagenesis^{15,16}, the genome of the UCD induced cellular cancer models was sequenced to uncover the genomic ramifications of UCD. An overall specific pyrimidine bias toward purines to pyrimidines (R->Y) compared to pyrimidines to purines (Y->R) point mutations on the DNA coding strand, was detected (FIG. 9). Furthermore, the TCGA data was interrogated and demonstrated that altered expression of genes encoding UC proteins was significantly associated with increased purine to pyrimidine transversion mutations in the DNA coding strand in many cancer types, denoted herein as (PTMB) (FIG. 6B, Table 1 hereinabove). Importantly, this association remained significant by controlling for the complementary pyrimidine to purine mutation (on the coding strand) in all cancer samples combined (FIG. 7B); and across individual cancer types (FIG. 7C). Interestingly, relative to samples with normal UC activity, in UCD samples the purine to pyrimidine mutations have a greater tendency to be non-synonymous, i.e. they change the encoded amino acid (FIG. 6C), suggesting that a shift toward pyrimidine mutation in UCD samples may confer a fitness advantage to the tumor. Indeed, the elevated purine to pyrimidine mutations associated with UCD persisted also at the mRNA level, as observed via the analysis of DNA and RNA sequences of 18 breast cancers samples (FIG. 7D).

Furthermore, proteomic analysis of 18 breast cancer tumors¹⁷ showed that all non-synonymous mutations identified at the DNA level persisted at the protein level, affirming that these mutations indeed induce the respective amino acid changes (FIG. 7E). Of note, the expression levels of the UC genes SLC25A13, SLC25A15 and CAD were among the top 10% of genes associated with the purine to pyrimidine mutation rates in cancer (FIG. 7F). Finally, the increased purine to pyrimidine mutation rate was associated with patient survival, independent of the rate of overall mutations (FIG. 6D).

Together, these results demonstrate that UCD induces a specific pyrimidine-rich transversion mutational bias signature in cancer that propagates from the DNA to mRNA to protein levels and is associated with patients' survival.

### Example 5

### UCD is Associated with Better Response to Immune Modulating Therapies

UCD-elicited pyrimidine-rich transversion mutational bias (PTMB) could result in the presentation of neo-antigens in tumor cells. Due to the outstanding relevance of this phenomenon for immunotherapy (Topalian et al., 2016), UCD and PTMB effects on the efficacy of immune checkpoint therapy (ICT) was evaluated. To this end, the transcriptomics of published data of melanoma patients treated with ICT (Van Allen et al., 2015), (Hugo et al., 2016) was analyzed and the UCD scores of the tumors were computed (where the gene expression of the 6 UC genes were available). Interestingly, responders to both anti-PD1 (Hugo et al., 2016) and anti-CTLA4 (Van Allen et al., 2015) therapy, had significantly higher UCD-scores than non-responders (FIG. 10A), and interestingly, this separation was higher than that seen using CAD expression levels (FIG. 11A). Following, a large exome sequencing cohort of patients treated with anti-PD1 (Roh et al., 2017) was analyzed, and indeed PTMB was found to be a stronger predictor of response to anti-PD1 therapy than mutational load (FIG. 10B).

To learn more about the potential mechanisms underlying the increased ICT response associated with UCD and PTMB, an HLA peptidomics analysis was performed on the genetically engineered UCD cancer cells having high PTMB levels (shown in FIGS. 2F and 8). It was found that the presentation of more neo-antigens with PTMB may be one factor that contributes to immunogenicity (Table 2 hereinbelow). Additionally, UCD could contribute to the immunogenicity through the presentation of more abundant and hydrophobic peptides (FIGS. 11B-C), which are known to incur stronger immunogenicity (Chowell et al., 2015); and highly hydrophobic peptides were found to be significantly more abundant than expected in UCD but not in control cells (FIG. 11D). Notably in this context, analysing the codon table of amino acids, revealed that R->Y mutations are significantly more likely to generate hydrophobic amino acids than other types of point mutations (Fisher exact test P<9.5E-5, odd ratio=2.67).

Taken together, these findings testify that the association of UCD to higher ICT efficacy is likely due to its combined effects of potentially generating PTMB-linked neo-antigens and perhaps more importantly, by generating more abundant and hydrophobic HLA-bound peptides.

Following, UCD and PTMB was induced in a syngeneic mouse model of colon cancer by knocking down ASS1. This UC perturbation resulted in larger tumors in vivo (FIGS. 12A-C), as was expected given the increased proliferation observed in UCD induced cancer cell-lines. Notably, the ASS1 perturbed tumors were significantly more sensitive to anti-PD1-based ICT than the unpertufbed ones (FIG. 10C). This increased therapeutic response was associated with enhanced specific infiltration of CD8 cytotoxic T-cells and not CD4 helper-T cells, as found in other studies (Wei et al., 2017) (FIGS. 10D and 12D). Notably, the response to anti-PD1 treatment was more efficient in mice bearing the ASS1 knockdown tumors compared to mice bearing unperturbed control tumors, reflected by a significantly attenuated progression of the tumor (FIGS. 10E and 12E).

TABLE 2
Identities of neo-antigens in UC-perturbed cancer cell lines

| | | R→Y | | | | | | Y→R | | | | | |
|---|---|---|---|---|---|---|---|---|---|---|---|---|---|
| UC | | | | Petides in | SEQ | | SEQ | | | | SEQ | | SEQ |
| Perturb- | | | Transcript_ | vector | ID | Peptides in | ID | | Transcript_ | Untreated | ID | Treated | ID |
| ation | Line | Gene | ID | control | NO | UCD cells | NO | Gene | ID | Peptide | NO | Peptide | NO |
| Citrin | Lox | HLA- | NM_002125 | GRPDAEY | 1 | GRPDDEY | 9 | IVL | NM_005547 | ELSEQQEGQL | 24 | ELSEQQEGQL | 26 |
| OE | | DRB5 | | | | | | | | | | | |
| | | PNPLA | NM_025225 | VCSCFIPF | 2 | VCSCFMPF | 10 | CALR | NM_004343 | KEEEEAEDK | 25 | KEEEEAEDK | 27 |
| | | 3 | | | | | | | | | | | |
| | | TPSD1 | NM_012217 | ALPVLASPAY | 3 | VLPVLASLAY | 11 | | | | | | |
| OTC | Hepg2 | HLA- | NM_002124 | QPKRECHF | 4 | QLKRECQF | 12 | | | | | | |
| KO | | DRB1 | | | | QPMWECQF | 13 | | | | | | |
| | | | | | | QHKMECQF | 14 | | | | | | |
| | | HLA- | NM_005514 | TAADTAAQITQR | 5 | TAADRAAQITPG | 15 | | | | | | |
| | | B | | | | TAADTAAQVTPG | 16 | | | | | | |
| | | | | | | TAADTGAQITPG | 17 | | | | | | |
| | | TPSD1 | NM_012217 | ALPVLASPAY | 6 | VLPVLASLAY | 18 | | | | | | |
| ASS1 | U2os | HLA- | NM_002124 | AVTELGRPDAEY | 7 | AATELGRPDAEH | 19 | | | | | | |
| KO | | DRB1 | | | | AATKLGRPDAEH | 20 | | | | | | |
| | | | | | | AATELGRPNAEH | 21 | | | | | | |
| | | | | | | AATELGRPDAQH | 22 | | | | | | |
| | | HLA- | NM_002125 | EDRRAAVDT | 8 | EETRAEVDT | 23 | | | | | | |
| | | DRB5 | | | | | | | | | | | |

*Three different human cancer cell lines, melanoma (LOX), hepatocellular carcinoma (HEPG2) and osteosarcoma (U2OS), induced with different UCD generated more neo antigens. The neo-antigens pulled down with HLA following specific UC perturbation in different cancers show they are enriched with R→Y mutation.

Taken together, the data reveals an oncogenic metabolic rewiring that maximizes the use of nitrogen by cancer cells and has diagnostic and prognostic values. Specifically, UCD was shown to be a common event in cancer which enhances nitrogen anabolism to pyrimidines by supplementing CAD with the three substrates needed for its function, supporting cell proliferation and mutagenesis, and correlating with survival risk. Moreover, the data reveals the hitherto unknown direct link between metabolic alterations in cancer, changes in nitrogen composition in biofluids and a genome-wide shift in mutational bias toward pyrimidines, generating metabolic and mutational signatures which encompass a persistent disruption in purine to pyrimidine nucleotide balance. The pyrimidine-rich transversion mutational bias propagates from the DNA to RNA and protein levels, leading to the generation of peptides with increased predicted immunogenicity, enhancing the response to immune-modulation therapy independently of mutational load both in mouse models and in patient correlative studies (FIG. 10F).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### REFERENCES

### Other References are Cited Throughout the Application

1 Valle D, B. A., Vogelstein B, Kinzler K W, Antonarakis S E, Ballabio A, Gibson K, Mitchell G. The Online Metabolic and Molecular Bases of Inherited Disease(McGraw-Hill, 2009).

2 Chaerkady, R. et al. A quantitative proteomic approach for identification of potential biomarkers in hepatocellular carcinoma. J Proteome Res 7, 4289-4298, doi:10.1021/pr800197z (2008).

3 Lee, Y. Y. et al. Overexpression of CPS1 is an independent negative prognosticator in rectal cancers receiving concurrent chemoradiotherapy. Tumour Biol 35, 11097-11105, doi:10.1007/s13277-014-2425-8 (2014).

4 Syed, N. et al. Epigenetic status of argininosuccinate synthetase and argininosuccinate lyase modulates autophagy and cell death in glioblastoma. Cell Death Dis 4, e458, doi:10.1038/cddis.2012.197 (2013).

5 Rabinovich, S. et al. Diversion of aspartate in ASS1-deficient tumours fosters de novo pyrimidine synthesis. Nature, doi:10.1038/nature15529 (2015).

6 Wise, D. R. & Thompson, C. B. Glutamine addiction: a new therapeutic target in cancer. Trends Biochem Sci 35, 427-433, doi:10.1016/j.tibs.2010.05.003 (2010).

7 van Kuilenburg, A. B., van Maldegem, B. T., Abeling, N. G., Wijburg, F. A. & Duran, M. Analysis of pyrimidine synthesis de novo intermediates in urine during crisis of a patient with ornithine transcarbamylase deficiency. Nucleosides Nucleotides Nucleic Acids 25, 1251-1255, doi:10.1080/15257770600894634 (2006).

8 Salerno, C. et al. Determination of urinary orotic acid and uracil by capillary zone electrophoresis. J Chronatogr B Biomed Sci Appl 734, 175-178 (1999).

9 van de Logt, A. E., Kluijtmans, L. A., Huigen, M. C. & Janssen, M. C. Hyperammonemia due to Adult-Onset N-Acetylglutamate Synthase Deficiency. JIMD Rep 31, 95-99, doi:10.1007/8904_2016_565 (2017).

10 Gerrits, G. P. et al. Argininosuccinic aciduria: clinical and biochemical findings in three children with the late onset form, with special emphasis on cerebrospinal fluid findings of amino acids and pyrimidines. Neuropediatrics 24, 15-18, doi:10.1055/s-2008-1071506 (1993).

11 Brosnan, M. E. & Brosnan, J. T. Orotic acid excretion and arginine metabolism. J Nutr 137, 1656S-1661S (2007).

12 DeVito, S. R., Ortiz-Riano, E., Martinez-Sobrido, L. & Munger, J. Cytomegalovirus-mediated activation of pyrimidine biosynthesis drives UDP-sugar synthesis to support viral protein glycosylation. Proc Nall Acad Sci USA 111, 18019-18024, doi:10.1073/pnas.1415864111 (2014).

13 Monks, A., Chisena, C. A. & Cysyk, R. L. Influence of ammonium ions on hepatic de novo pyrimidine biosynthesis. Arch Biochem Biophys 236, 1-10 (1985).

14 Wu, A. Tietz Clinical Guide to Laboratory Standards. 4th ed edn, (WB Saunders Company, 2006).

15 Kunz, B. A. Mutagenesis and deoxyribonucleotide pool imbalance. Mutat Res 200, 133-147 (1988).

16 Mathews, C. K. Deoxyribonucleotide metabolism, mutagenesis and cancer. Nat Rev Cancer 15, 528-539, doi:10.1038/nrc3981 (2015).

17 Zhang, H. et al. Integrated Proteogenomic Characterization of Human High-Grade Serous Ovarian Cancer. Cell 166, 755-765, doi:10.1016/j.cell.2016.05.069 (2016).

18 Gao, J. et al. Integrative analysis of complex cancer genomics and clinical profiles using the cBioPortal. Sci Signal 6, p11, doi:10.1126/scisignal.2004088 (2013).

19 Therneau, T. M. & Grambsch, P. M. Modeling survival data:extending the Cox model. (Springer, 2000).

20 McKenna, A. et al. The Genome Analysis Toolkit: a MapReduce framework for analyzing next-generation DNA sequencing data. Genome Res 20, 1297-1303, doi:10.1101/gr.107524.110 (2010).

21 Li, H. et al. The Sequence Alignment/Map format and SAMtools. Bioinformatics 25, 2078-2079, doi:10.1093/bioinformatics/btp352 (2009).

22 Duarte, N. C. et al. Global reconstruction of the human metabolic network based on genomic and bibliomic data. Proc Natl Acad Sci USA 104, 1777-1782, doi:10.1073/pnas.0610772104 (2007).

23 Terunuma, A. et al. MYC-driven accumulation of 2-hydroxyglutarate is associated with breast cancer prognosis. J Clin Invest 124, 398-412, doi:10.1172/JC171180 (2014).

24 Roessler, S. et al. Integrative genomic identification of genes on 8p associated with hepatocellular carcinoma progression and patient survival. Gastroenterology 142, 957-966 e912, doi: 10.1053/j.gastro.2011.12.039 (2012).

25 Tirosh, O. et al. The Transcription and Translation Landscapes during Human Cytomegalovirus Infection Reveal Novel Host-Pathogen Interactions. PLoS Pathog 11, e1005288, doi:10.1371/journal.ppat.1005288 (2015).

1-6. (canceled)

7. A method of treating cancer in a subject in need thereof, the method comprising:
(a) determining a level of urea and/or a pyrimidine synthesis metabolite in a biological sample of the subject; and wherein when a
(i) level of said urea below a predetermined threshold;
(ii) level of said pyrimidine synthesis metabolite above a predetermined threshold; and/or
(iii) ratio of said pyrimidine synthesis metabolite level to said urea level above a predetermined threshold;
is indicated
(b) treating said subject with a cancer therapy.

8. The method of claim 7, wherein said subject is diagnosed with cancer, wherein said (i), said (ii) and/or said (iii) is indicative of poor prognosis and wherein said
treating said subject with said cancer therapy is according to the prognosis.

9-10. (canceled)

11. The method of claim 7, wherein said biological sample is a biological fluid sample.

12. (canceled)

13. The method of claim 11, wherein said biological fluid sample is urine.

14. The method of claim 11, wherein said biological fluid sample is selected from the group consisting of blood, plasma and serum.

15. The method of claim 7, wherein said biological sample is cell-free.

16. (canceled)

17. The method of claim 7, wherein said predetermined threshold is at least 1.1 fold compared to a control sample.

18-23. (canceled)

24. The method of claim 7, wherein said cancer is selected from the group consisting of hepatic cancer, osteosarcoma, breast cancer, colon cancer, thyroid cancer, stomach cancer, lung cancer, kidney cancer, prostate cancer, head and neck cancer, bile duct cancer and bladder cancer.

25. The method of claim 7, wherein said cancer is selected from the group consisting of hepatic cancer, osteosarcoma, breast cancer and colon cancer.

26. (canceled)

27. The method of claim 7, wherein said cancer therapy comprises a therapy selected from the group consisting of L-arginine depletion, glutamine depletion, pyrimidine analogs, thymidylate synthase inhibitor and mammalian target of Rapamycin (mTOR) inhibitor.

28. The method of claim 7, wherein said cancer therapy comprises an immune modulation agent.

29. The method of claim 7, wherein said cancer therapy comprises an agent which induces a pyrimidines to purines nucleotide imbalance.

30. The method of claim 28, wherein said immune modulation agent comprises anti-PD1.

31. The method claim 28, wherein said immune modulation agent comprises anti-CTLA4.

32. The method of claim 29, wherein said agent which induces a pyrimidines to purines nucleotide imbalance comprises an anti-folate agent.

33. The method of claim 32, wherein said anti-folate agent comprises methotrexate.

34. The method of claim 7, wherein said pyrimidine synthesis metabolite is selected from the group consisting of Uracil, Thymidine, Orotic acid and Orotidine.


SEQUENCE LISTING (27 sequences)

SEQ ID NO: 1 - protein, 7 aa, Artificial sequence
  neo-antigens peptide sequence
GRPDAEY

SEQ ID NO: 2 - protein, 8 aa, Artificial sequence
  neo-antigens peptide sequence
VCSCFIPF

SEQ ID NO: 3 - protein, 10 aa, Artificial sequence
  neo-antigens peptide sequence
ALPVLASPAY

SEQ ID NO: 4 - protein, 8 aa, Artificial sequence
  neo-antigens peptide sequence
QPKRECHF

SEQ ID NO: 5 - protein, 12 aa, Artificial sequence
  neo-antigens peptide sequence
TAADTAAQITQR

SEQ ID NO: 6 - protein, 10 aa, Artificial sequence
  neo-antigens peptide sequence
ALPVLASPAY

SEQ ID NO: 7 - protein, 12 aa, Artificial sequence
  neo-antigens peptide sequence
AVTELGRPDAEY

SEQ ID NO: 8 - protein, 9 aa, Artificial sequence
  neo-antigens peptide sequence
EDRRAAVDT

SEQ ID NO: 9 - protein, 7 aa, Artificial sequence
  neo-antigens peptide sequence
GRPDDEY

SEQ ID NO: 10 - protein, 8 aa, Artificial sequence
  neo-antigens peptide sequence
VCSCFMPF

SEQ ID NO: 11 - protein, 10 aa, Artificial sequence
  neo-antigens peptide sequence
VLPVLASLAY

SEQ ID NO: 12 - protein, 8 aa, Artificial sequence
  neo-antigens peptide sequence
QLKRECQF

SEQ ID NO: 13 - protein, 8 aa, Artificial sequence
  neo-antigens peptide sequence
QPMWECQF

SEQ ID NO: 14 - protein, 8 aa, Artificial sequence
  neo-antigens peptide sequence
QHKMECQF

SEQ ID NO: 15 - protein, 12 aa, Artificial sequence
  neo-antigens peptide sequence
TAADRAAQITPG

SEQ ID NO: 16 - protein, 12 aa, Artificial sequence
  neo-antigens peptide sequence
TAADTAAQVTPG

SEQ ID NO: 17 - protein, 12 aa, Artificial sequence
  neo-antigens peptide sequence
TAADTGAQITPG

SEQ ID NO: 18 - protein, 10 aa, Artificial sequence
  neo-antigens peptide sequence
VLPVLASLAY

SEQ ID NO: 19 - protein, 12 aa, Artificial sequence
  neo-antigens peptide sequence
AATELGRPDAEH

SEQ ID NO: 20 - protein, 12 aa, Artificial sequence
  neo-antigens peptide sequence
AATKLGRPDAEH

SEQ ID NO: 21 - protein, 12 aa, Artificial sequence
  neo-antigens peptide sequence
AATELGRPNAEH

SEQ ID NO: 22 - protein, 12 aa, Artificial sequence
  neo-antigens peptide sequence
AATELGRPDAQH

SEQ ID NO: 23 - protein, 9 aa, Artificial sequence
  neo-antigens peptide sequence
EETRAEVDT

SEQ ID NO: 24 - protein, 10 aa, Artificial sequence
  neo-antigens peptide sequence
ELSEQQEGQL

SEQ ID NO: 25 - protein, 9 aa, Artificial sequence
  neo-antigens peptide sequence
KEEEEAEDK

SEQ ID NO: 26 - protein, 10 aa, Artificial sequence
  neo-antigens peptide sequence
ELSEQQEGQL

SEQ ID NO: 27 - protein, 9 aa, Artificial sequence
  neo-antigens peptide sequence
KEEEEAEDK